(19) United States
(12) Patent Application Publication
(10) Pub. No.: US 2024/0122909 A1
(43) Pub. Date: Apr. 18, 2024

(54) TREATMENTS OF HEREDITARY ANGIOEDEMA

(71) Applicant: KALVISTA PHARMACEUTICALS LIMITED, Wiltshire (GB)

(72) Inventors: Edward Paul FEENER, North Reading, MA (US); Sally Louise MARSH, Wiltshire (GB); Andreas MAETZEL, Chapel Hill, NC (US); Michael David SMITH, Salt Lake City, UT (US); Christopher Martyn YEA, Wiltshire (GB)

(21) Appl. No.: 18/264,810

(22) PCT Filed: Feb. 9, 2022
(86) PCT No.: PCT/GB22/50350

§ 371 Date: Aug. 9, 2023

Related U.S. Application Data
(60) Provisional application No. 63/147,595, filed on Feb. 9, 2021.
(60) Provisional application No. 63/212,224, filed on Jun. 18, 2021.
(60) Provisional application No. 63/220,747, filed on Jul. 12, 2021.
(60) Provisional application No. 63/277,753, filed on Nov. 10, 2021.
(60) Provisional application No. 63/286,363, filed on Dec. 6, 2021.

(51) Int. Cl. A61K 31/444; A61K 9/20; A61P 7/10

(52) U.S. Cl. CPC A61K 31/444; A61K 9/2054; A61P 7/10

(57) ABSTRACT

The present invention relates to treatments of hereditary angioedema (HAE). In particular, the present invention provides on-demand treatments of hereditary angioedema (HAE) by orally administering a plasma kallikrein inhibitor to a patient in need thereof on-demand.

The present invention relates to treatments of hereditary angioedema (HAE). In particular, the present invention provides on-demand treatments of hereditary angioedema (HAE) by orally administering a plasma kallikrein inhibitor to a patient in need thereof on-demand.

### BACKGROUND TO THE INVENTION

Inhibitors of plasma kallikrein have a number of therapeutic applications, particularly in the treatment of hereditary angioedema.

Plasma kallikrein is a trypsin-like serine protease that can liberate kinins from kininogens (see K. D. Bhoola et al., “Kallikrein-Kinin Cascade”, Encyclopedia of Respiratory Medicine, p 483-493; J. W. Bryant et al., “Human plasma kallikrein-kinin system: physiological and biochemical parameters” Cardiovascular and haematological agents in medicinal chemistry, 7, p 234-250, 2009; K. D. Bhoola et al., Pharmacological Rev., 1992, 44, 1; and D. J. Campbell, “Towards understanding the kallikrein-kinin system: insights from the measurement of kinin peptides”, Brazilian Journal of Medical and Biological Research 2000, 33, 665-677). It is an essential member of the intrinsic blood coagulation cascade although its role in this cascade does not involve the release of bradykinin or enzymatic cleavage. Plasma prekallikrein is encoded by a single gene and can be synthesized in the liver, as well as other tissues. It is secreted by hepatocytes as an inactive plasma prekallikrein that circulates in plasma as a heterodimer complex bound to high molecular weight kininogen (HK) which is activated to give the active plasma kallikrein. This contact activation system (or contact system) can be activated by negatively charged surfaces that activate Factor XII (FXII) to Factor XIIa (FXIIa), by certain proteases e.g. plasmin (Hofman et al Clin Rev Allergy Immunol 2016), which may not require negative surfaces, or by misfolded proteins (Maas et al J Clinical Invest 2008). FXIIa mediates conversion of plasma prekallikrein to plasma kallikrein and the subsequent cleavage of high molecular weight kininogen (HK) to generate bradykinin, a potent inflammatory hormone. Kinins are potent mediators of inflammation that act through G protein-coupled receptors and antagonists of kinins (such as bradykinin receptor antagonists) have previously been investigated as potential therapeutic agents for the treatment of a number of disorders (F. Marceau and D. Regoli, Nature Rev., Drug Discovery, 2004, 3, 845-852).

Plasma kallikrein is thought to play a role in a number of inflammatory disorders. The major inhibitor of plasma kallikrein is the serpin C1 esterase inhibitor. Patients who present with a genetic deficiency in C1 esterase inhibitor suffer from hereditary angioedema (HAE) which results in intermittent swelling of face, hands, throat, gastro-intestinal tract and genitals. Blisters formed during acute episodes contain high levels of plasma kallikrein which cleaves high molecular weight kininogen (HK) liberating bradykinin leading to increased vascular permeability. Treatment with a large protein plasma kallikrein inhibitor has been shown to effectively treat HAE by preventing the release of bradykinin which causes increased vascular permeability (A. Lehmann “Ecallantide (DX-88), a plasma kallikrein inhibitor for the treatment of hereditary angioedema and the prevention of blood loss in on-pump cardiothoracic surgery” Expert Opin. Biol. Ther. 8, p 1187-99).

Hereditary angioedema is a rare inherited disorder characterised by recurrent acute attacks where fluids accumulate outside of the blood vessels, blocking the normal flow of blood or lymphatic fluid and causing rapid swelling of tissues such as in the hands, feet, limbs, face, intestinal tract, or airway. “Hereditary angioedema” can thus be defined as any disorder characterised by recurrent episodes of bradykinin-mediated angioedema (e.g. severe swelling) caused by an inherited dysfunction/fault/mutation. There are currently three known categories of HAE: (i) HAE type 1, (ii) HAE type 2, and (iii) normal C1 inhibitor HAE (normal C1-Inh HAE). However, the HAE field is developing quickly so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Hoemost. 2019 January; 17(1):183-194. doi: 10.1111/jth.14325).

Acute HAE attacks (shortened to “HAE attacks”) normally progress through three key clinically distinct stages: an initial prodromal stage (that can typically last for up to 12 hours), followed by a swelling stage, and then an absorption stage. A majority of HAE attacks announce themselves with prodromal symptoms. Two thirds of prodromes appeared less than 6 hours before a HAE attack and no prodromes occur more than 24 hours before a HAE attack (Magerl et al. Clinical and Experimental Dermatology (2014) 39, pp 298-303). For example, the following prodromal symptoms may start to be observed: a slight swelling (particularly affecting the face and neck), a typical type of abdominal pain, a typical reddening of the skin called “Erythema marginatum”. An attack is fully developed when it has reached maximum swelling and maximum expression of pain (e.g. abdominal attack), discomfort (e.g. peripheral attack) or threat to life (e.g. laryngeal attack). Once the attack has reached its peak, the subsequent time period to normalization is determined by the time it takes for the swelling to disappear and the liquid that has penetrated the tissues to be reabsorbed.

Synthetic and small molecule plasma kallikrein inhibitors have been described previously, for example by Garrett et al. (“Peptide aldehyde . . . ” J. Peptide Res. 52, p 62-71 (1998)), T. Griesbacher et al. (“Involvement of tissue kallikrein but not plasma kallikrein in the development of symptoms mediated by endogenous kinins in acute pancreatitis in rats” British Journal of Pharmacology 137, p 692-700 (2002)), Evans (“Selective dipeptide inhibitors of kallikrein” WO03/076458), Szelke et al. (“Kininogenase inhibitors” WO92/04371), D. M. Evans et al. (Immunolpharmacology, 32, p 115-116 (1996)), Szelke et al. (“Kininogen inhibitors” WO95/07921), Antonsson et al. (“New peptides derivatives” WO94/29335), J. Corte et al. (“Six membered heterocycles useful as serine protease inhibitors” WO2005/123680), J. Stürzbecher et al. (Brazilian J. Med. Biol. Res 27, p 1929-34 (1994)), Kettner et al. (U.S. Pat. No. 5,187,157), N. Teno et al. (Chem. Pharm. Bull. 41, p 1079-1090 (1993)), W. B. Young et al. (“Small molecule inhibitors of plasma kallikrein” Bioorg. Med. Chem. Letts. 16, p 2034-2036 (2006)), Okada et al. (“Development of potent and selective plasmin and plasma kallikrein inhibitors and studies on the structure-activity relationship” Chem. Pharm. Bull. 48, p 1964-72 (2000)), Steinmetzer et al. (“Trypsin-like serine protease inhibitors and their preparation and use” WO08/049595), Zhang et al. (“Discovery of highly potent small molecule kallikrein inhibitors” Medicinal Chemistry 2, p 545-553 (2006)), Sinha et al. (“Inhibitors of plasma kallikrein” WO08/016883), Shigenaga et al. (“Plasma Kallikrein Inhibitors” WO2011/118672), and Kolte et al. (“Biochemical characterization of a novel high-affinity and specific kallikrein inhibitor”, British Journal of Pharmacology (2011), 162(7), 1639-1649). Also, Steinmetzer et al. (“Serine protease inhibitors” WO2012/004678) describes cyclized peptide analogs which are inhibitors of human plasmin and plasma kallikrein.

As explained above, HAE can manifest in patients who present with a genetic deficiency or dysfunction in C1 esterase inhibitor. Thus, some of the current treatments of HAE involve administering a C1 esterase inhibitor to normalise the deficiency or dysfunction in C1 esterase inhibitor. Such treatments can be prophylactic (i.e. administered in the absence of acute HAE attack symptoms to prevent/reduce the likelihood of an acute HAE attack) and/or acute treatments (i.e. administered when acute HAE attack symptoms are noticed to try to stop or reduce the severity of the acute HAE attack).

Cinryze® and Haegarda® contain a C1 esterase inhibitor and are indicated to prevent acute HAE attacks (i.e. prophylactic treatment). Treatment with Cinryze® requires the preparation of a solution from a powder, which is then injected every 3 or 4 days. Similarly, treatment with Haegarda® requires the preparation of a solution from a powder, which is then injected twice a week. It is not always possible for a patient to self-administer these treatments, and if this is the case, the patient is required to visit a clinic for treatment. Thus, both of these prophylactic treatments suffer from high patient burden. Additionally, the FDA packet insert for Haegarda® states that it “should not be used to treat an acute HAE attack”, and therefore a patient may require additional therapy if a HAE attack develops.

Berinert® and Ruconest® contain a C1 esterase inhibitor and are indicated to treat acute HAE attacks. Both of these treatments also involve the preparation of an injectable solution followed by injection. This process can be burdensome on the patient, especially when the patient is suffering from an acute HAE attack. Self-administration of the dosage amount is also not always possible, and if it is not, administration of the drug can be substantially delayed thus increasing the severity of the acute HAE attack for the patient.

Selective plasma kallikrein inhibitors approved for medical use in the treatment of HAE include Kalbitor® (active substance ecallantide) and Takhzyro® (active substance lanadelumab). Both treatments are formulated as solutions for injection. Ecallantide is a large protein plasma kallikrein inhibitor that presents a risk of anaphylactic reactions. Indeed, the EU marketing authorisation application for Kalbitor® has recently been withdrawn because the benefits of Kalbitor® are said to not outweigh its risks. Lanadelumab is a recombinant fully human IgG1 kappa light chain monoclonal antibody. Reported adverse reactions of treatment with lanadelumab include hypersensitivity, injection site pain, injection site erythema, and injection site bruising. The authorised EMA label for Takhzyro® (active substance lanadelumab) states that it “is not intended for treatment of acute HAE attacks” and that “in case of a breakthrough HAE attack, individualized treatment should be initiated with an approved rescue medication”. Also, as injections, both of these treatments involve a high patient burden.

Berotralstat (BCX7353) has been approved in some countries for preventative treatment of HAE (not on-demand treatment), e.g. under the brand name Orladeyo®. Hwang et al. (Immunotherapy (2019) 11(17), 1439-1444) states that higher doses were associated with more gastrointestinal adverse effects indicating increased toxicity at higher levels.

Other plasma kallikrein inhibitors known in the art are generally small molecules, some of which include highly polar and ionisable functional groups, such as guanidines or amidines. Recently, plasma kallikrein inhibitors that do not feature guanidine or amidine functionalities have been reported. For example Brandl et al. (“N-((6-amino-pyridin-3-yl)methyl)-heteroaryl-carboxamides as inhibitors of plasma kallikrein” WO2012/017020), Evans et al. (“Benzylamine derivatives as inhibitors of plasma kallikrein” WO2013/005045), Allan et al. (“Benzylamine derivatives” WO2014/108679), Davie et al. (“Heterocyclic derivates” WO2014/188211), and Davie et al. (“N-((het)arylmethyl)-heteroaryl-carboxamides compounds as plasma kallikrein inhibitors” WO2016/083820).

The applicant has developed a novel series of compounds that are inhibitors of plasma kallikrein, which are disclosed in WO2016/083820 (PCT/GB2015/053615). These compounds demonstrate good selectivity for plasma kallikrein. One such compound is N-[(3-fluoro-4-methoxypyridin-2-yl)methyl]-3-(methoxymethyl)-1-({4-[(2-oxopyridin-1-yl)methyl]phenyl}methyl)pyrazole-4-carboxamide (depicted as the compound of Formula A below). Treatments of HAE comprising the compound of Formula A are discussed in WO2020/249977.

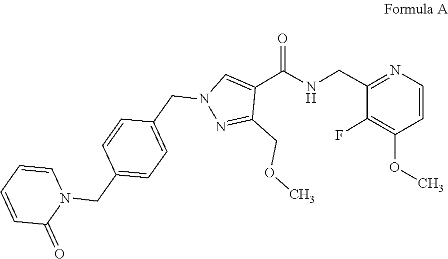 [SMILES: COCc1nn(Cc2ccc(Cn3ccccc3=O)cc2)cc1C(=O)NCc1nccc(OC)c1F]

There remains the need for approved treatments of HAE that are less burdensome on the patient to improve patient compliance. Current on-demand therapies are injectable treatments, and patients must have confidence in any alternative (non-injectable) therapy if they are to move away from their current therapy to such alternative therapies.

### Definitions

The terms “attack of HAE”, “HAE attack”, and “attack”, etc. are used interchangeably herein. The term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

The term “consecutive time points” when used in the context of “symptom relief” can mean an assessment as described in Table 3. Specifically, “consecutive time points” can mean an assessment separated by 30 mins (particularly within 0-4 hours after administration of the compound). “Consecutive time points” can also mean an assessment separated by 1 hour (particularly within 4-12 hours after administration of the compound). “Consecutive time points” can also mean an assessment separated by 3 hour (particularly within 12-24 hours after administration of the compound).

In any of the treatments of the invention described herein, the term “compound of Formula A” or “compound” is shorthand for “compound of Formula A (or a pharmaceutically acceptable salt and/or solvate thereof)”. The term “solvate” is used herein to describe a molecular complex comprising the compound of the invention and a one or more pharmaceutically acceptable solvent molecules, for example, ethanol or water. The term “hydrate” is employed when the solvent is water and for the avoidance of any doubt, the term “hydrate” is encompassed by the term “solvate”.

In any of the treatments of the invention described herein, the term “pharmaceutically acceptable salt” means a physiologically or toxicologically tolerable salt and includes pharmaceutically acceptable acid addition salts. For example (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

The compound of Formula A is meant to include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention.

“On-demand” treatment, in the context of HAE, means that the compound of Formula A is administered upon need of therapy in connection with one specific HAE attack. “On-demand” does not require the administration of the compound of Formula A continuously at regular intervals (e.g. once a week, twice a week, etc.) irrespective of an instance of an HAE attack. This is in contrast to some other known treatments of HAE (e.g. treatments with Cinryze® and Haegarda®, as described above) that require continuous and regular dosing for therapy. Instead, in treatments of the invention, the compound of Formula A is taken when the patient requires fast-acting therapeutic effects. These are discussed below in more detail.

In any of the treatments of the invention described herein, the patient is preferably a human. HAE is a hereditary disease and patients of all ages can suffer from HAE attacks. Accordingly, the human patient can be a child (ages 0 to 18 years) or an adult (18 years old or older). Specifically, the patient can be aged 12 years and above. The patient can also be aged 2 years and above.

### DESCRIPTION OF THE INVENTION

To date, there are no authorised on-demand oral treatments of HAE, with all of the authorised treatments being injectable. HAE attacks resolve faster and are shorter after early treatment (Maurer M et al. PLoS ONE 2013; 8(2): e53773. doi:10.1371/journal.pone.0053773) and thus early intervention when an attack is expected, or ongoing, is essential to desirably manage the disease. Injectable treatments suffer from late dosing because the patient may need to prepare the dosage form or even travel to hospital for treatment. Therefore, HAE treatment is often undermined by late dosing caused by the high burden on the patient. Indeed, Maurer M et al. explains that more than 60% of patients administer their HAE injectable more than one hour after the onset of an attack. Without wishing to be bound by theory, it is thought that HAE injectable treatments suffer from late dosing for reasons such as inconvenience (self-administration is not always possible), pain (both during and after the injection), and hope (rather than treat, patients frequently will just hope for a less severe attack). Nevertheless, there is currently a reliance on injectable therapies for on-demand treatment of HAE patients, and further patient confidence that oral on-demand therapies can replace injectable therapies would be desirable. The present invention aims to solve this problem.

The efficacy data from the phase 2 study described in Example 6 confirm that treatment with the compound of Formula A rapidly suppresses circulating plasma kallikrein, halts attack progression, reduces symptoms and improves patient well-being. These are statistically significant results. Being an oral treatment, the compound of Formula A enables early intervention that can prevent HAE attacks from ever reaching high severity, which can lead to improved treatment outcomes (e.g. faster symptom relief). Patients can feel better and symptoms can resolve quickly with treatment with the compound of Formula A. Indeed, the efficacy profile demonstrated by the compound of Formula A is fast and comparable with current approved injectable products, and is also generally safe and well tolerated. These efficacy results can provide a patient with further confidence that they are able to dose on-demand, and specifically, dose on-demand with an oral treatment, and still achieve efficacious treatment.

The invention provides a method for treating an attack of hereditary angioedema (HAE) on-demand comprising: orally administering the compound of Formula A (or a pharmaceutically acceptable salt and/or solvate thereof) to a patient in need thereof on-demand, wherein symptom relief of the attack starts within 3 hours of administering the compound of Formula A (or a pharmaceutically acceptable salt and/or solvate thereof). The term “symptom relief” in this context can mean that the attack is rated as “a little better” or higher for two consecutive time points when assessed according to the 7-point transition questions (7TQ). 7TQ (or PGI-C) is a known index in the art that can be used to score the progression of an HAE attack and to report attacks as “much better”, “better”, “a little better”, “no change”, “a little worse”, “worse”, or “much worse”.

When assessed using the 7TQ, treatments of the invention can lead to symptom relief starting between about 0.6 and 3 hours after administering the compound. Symptom relief can start between about 1 and 3 hours after administering the compound. Symptom relief can start between 1.5 and 3 hours after administering the compound. Symptom relief can start between 1.5 and 1.8 hours after administering the compound. Symptom relief can start at about 1.6 hours after administering the compound.

Alternatively, or additionally, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein symptom relief of the attack starts between about 1.5 and 9 hours after administering the compound of Formula A (or a pharmaceutically acceptable salt and/or solvate thereof). The term “symptom relief” in this context can mean that a 50% reduction in composite VAS score occurred for three consecutive time points. The composite VAS score is a known assessment of the severity of HAE attack symptoms (abdominal pain, skin pain and skin swelling), each assessed on a 100 mm visual analogue scale (VAS) anchored at 0 (none) and 100 (very severe).

When assessed using the composite VAS score, treatments of the invention can lead to symptom relief starting between 2.5 and 9 hours after administering the compound. Symptom relief can start between 3 and 9 hours after administering the compound. Symptom relief can start between 4 and 7 hours after administering the compound. Symptom relief can start at about 6 hours after administering the compound.

Alternatively, or additionally, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the treatment halts the progress of the attack such that the symptoms of the attack do not worsen within a 12 hour period after administration of the compound of Formula A (or a pharmaceutically acceptable salt and/or solvate thereof). In this context, “do not worsen” can mean that the attack did not worsen by one level or more on the 5LS within the 12 hours of administering the compound. 5LS (also called PGI-S) is a known scale in the art (see e.g. Allergy Asthma Proc. 2018 Jan. 1; 39(1):74-80. doi: 10.2500/aap.2018.39.4095) that can be used to report the severity of HAE attacks and for example can be used to report attacks as “none”, “mild”, “moderate”, “severe” or “very severe”. Treatment according to the invention can halt the progress of an attack of hereditary angioedema (HAE) such that the symptoms of the attack do not worsen within a 24 hour period after administration of the compound of Formula A (or a pharmaceutically acceptable salt and/or solvate thereof). In this context, “do not worsen” can mean that the attack did not worsen by one level or more on the 5LS within the 24 hours of administering the compound.

Alternatively, or additionally, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the treatment improves the severity of the attack to “none” (when assessed according to the 5LS). Treatment according to the invention can improve the severity of the attack to “none” within 24 hours of administering the compound. Treatment according to the invention can improve the severity of the attack to “none” within 21 hours of administering the compound. Treatment according to the invention can improve the severity of the attack to “none” within 18 hours of administering the compound. Treatment according to the invention can improve the severity of the attack to “none” within 15 hours of administering the compound. Treatment according to the invention can improve the severity of the attack to “none” within 12 hours of administering the compound. Treatment according to the invention can improve the severity of the attack to “none” within 9 hours of administering the compound. Treatment according to the invention can improve the severity of the attack to “none” within 6 hours of administering the compound. Treatment according to the invention can improve the severity of the attack to “none” within 4 hours of administering the compound. Treatment according to the invention can improve the severity of the attack to “none” within 2 hours of administering the compound.

Alternatively, or additionally, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the treatment improves the severity of the attack by one level or more when assessed according to the 5LS. “Improves the severity” means reducing severity of the attack i.e. the symptoms of the attack get better. Treatment according to the invention can improve the severity of the attack by one level or more when assessed according to the 5LS within 24 hours of administering the compound. Treatment according to the invention can improve the severity of the attack by one level or more when assessed according to the 5LS within 18 hours of administering the compound. Treatment according to the invention can improve the severity of the attack by one level or more when assessed according to the 5LS within 12 hours of administering the compound. Treatment according to the invention can improve the severity of the attack by one level or more when assessed according to the 5LS within 9 hours of administering the compound. Treatment according to the invention can improve the severity of the attack by one level or more when assessed according to the 5LS within 6 hours of administering the compound.

Alternatively, or additionally, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the treatment reduces the need for a second treatment of the HAE attack within 4 hours of taking the compound of Formula A. The treatment can reduce the need for a second treatment of the HAE attack within 6 hours of taking the compound of Formula A. The treatment can reduce the need for a second treatment of the HAE attack within 8 hours of taking the compound of Formula A. The treatment can reduce the need for a second treatment of the HAE attack within 12 hours of taking the compound of Formula A. The treatment can reduce the need for a second treatment of the HAE attack within 24 hours of taking the compound of Formula A. The treatment can reduce the need for a second treatment attack at all to resolve the attack. “To resolve the attack” means that the HAE attack clears without further treatment.

More specifically, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the treatment avoids the need for a second treatment of the HAE attack within 4 hours of taking the compound of Formula A. The treatment can avoid the need for a second treatment of the HAE attack within 6 hours of taking the compound of Formula A. The treatment can avoid the need for a second treatment of the HAE attack within 8 hours of taking the compound of Formula A. The treatment can avoid the need for a second treatment of the HAE attack within 12 hours of taking the compound of Formula A. The treatment can avoid the need for a second treatment of the HAE attack within 24 hours of taking the compound of Formula A. The treatment can avoid the need for a second treatment to resolve the attack.

Even more specifically, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the patient is not administered a second treatment of the HAE attack within 4 hours of taking the compound of Formula A. The patient may not be administered a second treatment within 6 hours of taking the compound of Formula A. The patient may not be administered a second treatment of the HAE attack within 8 hours of taking the compound of Formula A. The patient may not be administered a second treatment of the HAE attack within 12 hours of taking the compound of Formula A. The patient may not be administered a second treatment of the HAE attack within 24 hours of taking the compound of Formula A. The attack can be resolved without administering any second treatment to the patient.

As used herein, the “second treatment of the HAE attack” or “second treatment” is the administration of any dose of active pharmaceutical ingredient for treating an HAE attack that is not the initial dose of the compound. The second treatment can be an existing HAE treatment. For example, the “second treatment” can be pdC1INH, rhC1INH, icatibant, conestat alfa (Ruconest®), ecallantide, or second dosage amount of the compound of Formula A.

In the treatments of the invention, the compound of Formula A can be orally administered on-demand upon recognition of a symptom of an HAE attack. Although each HAE attack can be different in severity and in terms of the area affected, patients who suffer from HAE, medical professionals with knowledge of HAE, and carers of HAE patients are (and indeed the skilled person would be) astute in identifying symptoms of an HAE attack. These symptoms include, but are not limited to: swelling of tissues such as in the hands, feet, limbs, face, intestinal tract, and/or airway; fatigue; headache; muscle aches; skin tingling; abdominal pain; nausea; vomiting; diarrhoea; difficulty swallowing; hoarseness; shortness of breath; and/or mood changes. Thus, in some embodiments, administration of the compound of Formula A can occur upon recognition of at least one of the above symptoms.

The skilled person would also understand that “administered upon recognition of a symptom of a HAE attack” means that administration occurs as quickly as feasibly possible after the symptom of an HAE attack is recognised. For example, patients are expected to have the compound of Formula A easily and readily available at all times (most likely in the form of a pharmaceutically acceptable composition) to ensure that treatment can occur upon recognition of a symptom of a HAE attack. In other words, the treatment occurs on-demand. For example, the compound of Formula A can be administered within 1 hour of the symptom of an HAE attack being recognised, preferably within 30 minutes, within 20 minutes, within 10 minutes, or within 5 minutes of the symptom of an HAE attack being recognised.

If the symptom of an HAE attack is recognised in the prodromal phase, the compound of Formula A can be administered in the prodromal phase of an HAE attack. In these circumstances, the symptom recognised can be a slight swelling, in particular, a slight swelling affecting the face and neck. In addition, or in the alternative, the symptom can be abdominal pain, in particular, abdominal pain is considered to be characteristic of a HAE attack. In addition, or in the alternative, the symptom can be a reddening of the skin such as Erythema marginatum.

Treatments according to the invention can treat abdominal HAE attacks. Treatments according to the invention can treat peripheral HAE attacks.

As noted above, treatment in accordance with the invention can prevent an HAE attack from increasing in severity. In some circumstances, treatment can shorten the attack duration, and sometimes even halt the attack in its entirety. For instance, treatment can halt the progression of a peripheral HAE attack or an abdominal HAE attack. In some embodiments, treatment according to the invention can suppress the subsequent onset of swelling, sometimes completely, and in particular when treatment is initiated in the prodromal phase. In particular, in some embodiments, the HAE attack can be prevented from progressing into the swelling stage when the treatment is initiated in the prodromal phase.

In any of the above treatments, the symptom can be recognised by the patient. In any of the above treatments, the symptom can be recognised by a medical professional such as a medical professional with knowledge of HAE. In any of the above treatments, the symptom can be recognised by a carer of the patient.

Treatments according to the invention can reduce the proportion of HAE attacks that progress by one level or more on a 5-point Likert scale (5LS). Treatments according to the invention can reduce the proportion of HAE attacks that progress by one level or more on a 5LS within 12 hours of administering the compound. Treatments according to the invention can improve the resolution time of a HAE attack to “none” on a 5LS. 5LS is a known scale in the art (see e.g. Allergy Asthma Proc. 2018 Jan. 1; 39(1):74-80. doi: 10.2500/aap.2018.39.4095) that can be used to report the severity of HAE attacks and for example can be used to report attacks as “none”, “mild”, “moderate”, “severe” or “very severe”.

Treatments according to the invention can reduce the proportion of HAE attacks that are rated “worse” or “much worse” on a 7-point transition question (7TQ). Treatments according to the invention can increase the proportion of HAE attacks that are rated as “better” or “much better”. 7TQ is a known index in the art that can be used to score the progression of an HAE attack and to report attacks as “much better”, “better”, “a little better”, “no change”, “a little worse”, “worse”, or “much worse”.

The inventors have identified that patients who achieved a PGI-C (also known as the 7TQ) rating of “better” or higher within 24 hours were less likely to use rescue medication (i.e. less likely to take a second treatment of the HAE attack) and more likely to achieve attack resolution when assessed by the PGI-S or VAS.

Thus, and additionally or alternatively to other treatments of the invention, the invention also provides a method for treating an attack of hereditary angioedema (HAE) on-demand comprising: orally administering the compound of Formula A (or a pharmaceutically acceptable salt and/or solvate thereof) to a patient in need thereof on-demand, wherein the attack improves to a rating of “better” or higher between about 1.5 and 16 hours after administering the compound, when assessed using the 7TQ. More specifically, the attack improves to a rating of “better” or higher about 2.1 and 15 hours after administering the compound. More specifically, the attack improves to a rating of “better” or higher between about 2.5 and 10 hours after administering the compound. More specifically, the attack improves to a rating of “better” or higher between about 2.5 and 8 hours after administering the compound. More specifically, the attack improves to a rating of “better” or higher between about 3 and 8 hours after administering the compound. More specifically, the attack improves to a rating of “better” or higher between about 4 and 6 hours after administering the compound. More specifically, the attack improves to a rating of “better” or higher between about 4.5 and 5.5 hours after administering the compound. More specifically, the attack improves to a rating of “better” or higher at about 5 hours after administering the compound.

Thus, and additionally or alternatively to other treatments of the invention, the invention also provides a method for treating an attack of hereditary angioedema (HAE) on-demand comprising: orally administering the compound of Formula A (or a pharmaceutically acceptable salt and/or solvate thereof) to a patient in need thereof on-demand, wherein the attack improves to a rating of “better” or higher between about 1.5 and 16 hours after administering the compound, when assessed using the 7TQ over two consecutive time points. More specifically, a rating of “better” or higher can start between about 2.1 and 15 hours after administering the compound. More specifically, a rating of “better” or higher can start between about 2.5 and 10 hours after administering the compound. More specifically, a rating of “better” or higher can start between about 2.5 and 8 hours after administering the compound. More specifically, a rating of “better” or higher can start between about 3 and 8 hours after administering the compound. More specifically, a rating of “better” or higher can start between about 4 and 6 hours after administering the compound. More specifically, a rating of “better” or higher can start between about 4.5 and 5.5 hours after administering the compound. More specifically, a rating of “better” or higher can start at about 5 hours after administering the compound.

The inventors have identified that patients who achieved a PGI-C (also known as the 7TQ) rating of “A Little Better” or higher at 2 consecutive time points within 24 hours were less likely to use rescue medication (i.e. less likely to take a second treatment of the HAE attack) and more likely to achieve attack resolution.

Specifically, the present invention provides a method for treating an attack of hereditary angioedema (HAE) on-demand comprising: orally administering the compound of Formula A (or a pharmaceutically acceptable salt and/or solvate thereof) to a patient in need thereof on-demand, wherein symptom relief of the attack starts within 3 hours (e.g. between about 1 and about 3 hours, between about 1.5 and about 3 hours, between about 1.5 and about 1.8 hours, at about 1.6 hours) of administering the compound of Formula A (or a pharmaceutically acceptable salt and/or solvate thereof), wherein the term “symptom relief” in this context can mean that the attack is rated as “a little better” or higher for two consecutive time points when assessed according to the 7-point transition questions (7TQ), and wherein the treatment reduces the need for a second treatment of the HAE attack.

More specifically, the present invention provides a method for treating an attack of hereditary angioedema (HAE) on-demand comprising: orally administering the compound of Formula A (or a pharmaceutically acceptable salt and/or solvate thereof) to a patient in need thereof on-demand, wherein symptom relief of the attack starts within 3 hours (e.g. between about 1 and about 3 hours, between about 1.5 and about 3 hours, between about 1.5 and about 1.8 hours, at about 1.6 hours) of administering the compound of Formula A (or a pharmaceutically acceptable salt and/or solvate thereof), wherein the term “symptom relief” in this context can mean that the attack is rated as “a little better” or higher for two consecutive time points when assessed according to the 7-point transition questions (7TQ), and wherein the treatment avoids the need for a second treatment of the HAE attack.

Even more specifically, the present invention provides a method for treating an attack of hereditary angioedema (HAE) on-demand comprising: orally administering the compound of Formula A (or a pharmaceutically acceptable salt and/or solvate thereof) to a patient in need thereof on-demand, wherein symptom relief of the attack starts within 3 hours (e.g. between about 1 and about 3 hours, between about 1.5 and about 3 hours, between about 1.5 and about 1.8 hours, at about 1.6 hours) of administering the compound of Formula A (or a pharmaceutically acceptable salt and/or solvate thereof), wherein the term “symptom relief” in this context can mean that the attack is rated as “a little better” or higher for two consecutive time points when assessed according to the 7-point transition questions (7TQ), and wherein the patient is not administered a second treatment of the HAE attack.

Alternatively, or additionally, the invention provides a method for treating an attack of hereditary angioedema (HAE) on-demand comprising: orally administering the compound of Formula A (or a pharmaceutically acceptable salt and/or solvate thereof) to a patient in need thereof on-demand, wherein the treatment reduces (or preferably prevents) the progress of the attack. For instance, the treatment can prevent an attack from being rated as “a little worse” or higher on the PGI-C (also known as the 7TQ) over two consecutive time points. The treatment can prevent an attack from being rated as “a little worse” or higher on the PGI-C over two consecutive time points within 12 hours of administering the compound. More specifically, the treatment can prevent an attack from being rated as “a little worse” or higher on the PGI-C over two consecutive time points within 24 hours of administering the compound.

Any of the treatments of the invention may be used in combination with other treatments of HAE. In particular, the treatments described herein can be used as a “top-up” (or rescue medication) to another treatment of HAE e.g. a prophylactic treatment of HAE. The patient may be taking a prophylactic treatment of HAE with another drug and can use the on-demand treatments described herein to treat an acute HAE attack that was not prevented by the prophylactic treatment (so called “breakthrough attacks”). The prophylactic treatment of HAE can be selected from C1 inhibitor (such as Cinryze®, Haegarda®, Berinert®), lanadelumab, and berotralstat.

For instance, the invention can provide a method for treating HAE in a patient already taking a C1 inhibitor (such as Cinryze®, Haegarda®, Berinert®) for prophylaxis comprising: orally administering the compound of Formula A to the patient on-demand to treat a breakthrough attack (in accordance with any of the on-demand treatments described herein). Also provided is a method for treating HAE in a patient already taking lanadelumab for prophylaxis comprising: orally administering the compound of Formula A to the patient on-demand to treat a breakthrough attack (in accordance with any of the on-demand treatments described herein). Also provided is a method for treating HAE in a patient already taking berotralstat for prophylaxis comprising: orally administering the compound of Formula A to the patient on-demand to treat a breakthrough attack (in accordance with any of the on-demand treatments described herein).

The prophylactic treatment of HAE can also be 1-({4-[(5-fluoro-2-oxopyridin-1-yl)methyl]phenyl}methyl)-N-[(3-fluoro-4-methoxypyridin-2-yl)methyl]-3-(methoxymethyl)pyrazole-4-carboxamide (or a pharmaceutically acceptable salt and/or solvate thereof). 1-({4-[(5-fluoro-2-oxopyridin-1-yl)methyl]phenyl}methyl)-N-[(3-fluoro-4-methoxypyridin-2-yl)methyl]-3-(methoxymethyl)pyrazole-4-carboxamide is also known as “KVD824”. At the time of filing, KVD824 has completed phase 1 studies and has begun phase 2 studies (NCT05055258|EudraCT number: 2021-000136-59—the KOMPLETE™ clinical trial). In its phase 1 studies, concentrations of KVD824 were shown to provide a consistent pharmacokinetic profile over repeat dosing, and were maintained at concentrations above functional concentrations of C1 inhibitor and lanadelumab. Prophylactic treatments of HAE comprising KVD824 (or a pharmaceutically acceptable salt and/or solvate thereof) are also described in PCT/GB2021/052678, and KVD824's plasma kallikrein inhibitor activity is described in WO2019/106377 and WO2017/207983. Thus, the invention also provides a method for treating HAE in a patient already taking KVD824 (or a pharmaceutically acceptable salt and/or solvate thereof) for prophylaxis comprising: orally administering the compound of Formula A to the patient on-demand to treat a breakthrough attack (in accordance with any of the on-demand treatments described herein).

Dosing

In any of the treatments of the invention described herein, the compound of Formula A is orally administered in a therapeutically effective amount.

The compound of Formula A can be administered at an amount of between about 100 mg and about 1500 mg, about 300 mg to about 1800 mg, about 100 mg and about 1400 mg, about 200 mg and about 1200 mg, about 300 mg and about 1200 mg, about 600 mg and about 1200 mg, about 450 mg and about 900 mg, about 500 mg and about 1000 mg, about 450 mg and about 600 mg, about 500 mg and about 700 mg (more specifically, 600 mg), about 800 mg and about 1000 mg per day, about 900 mg and about 1400 mg (more specifically 1200 mg), or about 900 mg and about 1200 mg. The dosage amount can be 300 mg. The dosage amount can be 600 mg. The daily dosage amount can be 900 mg. In another specific embodiment, the daily dosage amount can be 1200 mg. The dosage amount is 1800 mg.

Preferably, the dosage amount is 600 mg. Preferably, the dosage amount is administered within 1 hour of the symptom of an HAE attack being recognised.

The dosage amount can also be 300 mg. Preferably, the dosage amount is administered within 1 hour of the symptom of an HAE attack being recognised.

Each dosage amount administered to the patient can be sub-divided into small unit dosage amounts. For example, the preferred 600 mg dosage amount of the compound can be sub-divided into two unit dosage amounts (e.g. two tablets), each comprising 300 mg of the compound.

The treatments of the invention involve oral administration. In any of the treatments of the invention, the compound of Formula A can be administered as an oral dosage form comprising the compound of Formula A and pharmaceutically acceptable excipients. The oral dosage form can be in the form of a tablet or a capsule. The oral dosage form can be a tablet. The oral dosage form can be a capsule.

The dosage form can be a tablet comprising microcrystalline cellulose as a diluent, croscarmellose sodium as a disintegrant, polyvinyl pyrrolidone as a binder, and optionally magnesium stearate as a lubricant. In a preferred tablet, the compound of Formula A comprises: (i) at least about 40 wt % of the tablet (more specifically about 40 wt % to about 60 wt %), compared to the total mass of the tablet; (ii) about 25 wt % to about 60 wt % of the diluent (more specifically about 25 wt % to about 40 wt %, compared to the total mass of the tablet; (iii) about 1 wt % to about 15 wt % of the disintegrant (more specifically about 2 wt % to about 6 wt %), compared to the total mass of the tablet; (iv) about 1 wt % to about 20 wt % of the binder (more specifically about 2 wt % to about 5 wt %), compared to the total mass of the tablet; and when present, (v) about 0.1 to about 5 wt % lubricant (more specifically about 0.1 wt % to about 1.5 wt %), compared to the total mass of the tablet. The dosage form can be a tablet containing 300 mg of the compound.

The tablet can further comprise extragranular excipients comprising: microcrystalline cellulose as an extragranular diluent, croscarmellose sodium as an extragranular disintegrant, polyvinyl pyrrolidone as an extragranular binder, and/or magnesium stearate as an extragranular lubricant.

The dosage forms described herein (e.g. the tablets) can be film coated, wherein the film coating can comprise one or more of hypromellose, lactose monohydrate, titanium dioxide and triacetin.

Further Specifics of the Treatments of the Invention

As shown herein, the compound of Formula A has a rapid onset of action. Specifically, the compound of Formula A is a potent inhibitor of plasma kallikrein activity and is highly effective at interrupting the contact activation system's positive feedback loop between plasma kallikrein, prekallikrein, Factor XII (FXII), and Factor XIIa (FXIIa). The pharmacokinetic and pharmacodynamic data provided herein demonstrate that these effects are shown quickly after oral administration of the compound of Formula A. Accordingly, the treatments of the invention are fast acting and are thus particularly suited to treating HAE on-demand.

As discussed above, the treatments of the invention are particularly advantageous when the concentration of the compound of Formula A is at least 500 ng/mL in plasma. A plasma concentration of at least 500 ng/mL can be observed following administration of a dosage amount of at least about 60 mg (more specifically, at least about 70 mg or about 80 mg) of the compound of Formula A.

The treatments according to the invention provide rapid protection from HK (high molecular weight kininogen) cleavage that are particularly suited to shortening the severity (or even halting) an ongoing HAE attack. As described here, the treatments according to the invention also have a prolonged pharmacodynamic effect. The pharmacodynamic effects of the compound of Formula A that are related to treating HAE include providing protection from HK cleavage, which as discussed above, can cause an HAE attack. For example, the compound of Formula A can provide protection from HK cleavage by at least (i) inhibiting plasma kallikrein, (ii) reducing cleavage of plasma prekallikrein, and/or (iii) reducing the generation of Factor XIIa from Factor XII.

The treatments according to the invention can provide protection from HK (high molecular weight kininogen) cleavage within one hour post-dosage amount, and in particular when the dosage amount of the compound of Formula A is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). The treatments according to the invention can provide protection from HK (high molecular weight kininogen) cleavage within 45 minutes post-dosage amount, or within 30 minutes post-dosage amount. Protection from HK (high molecular weight kininogen) cleavage can be determined by comparing HK levels in untreated plasma with HK levels in treated plasma i.e. plasma from subjects that have received a dosage amount of the compound of Formula A, and then activating the plasma with dextran sulfate to activate the contact system to induce HK cleavage. If the HK level in the treated plasma is above the HK level in the untreated plasma, then the HK has been protected from HK cleavage in the activated plasma.

The treatment can inhibit at least 80% of plasma kallikrein activity within 30 minutes post-dosage amount, and in particular when the dosage amount of the compound of Formula A is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). In some embodiments of the invention, the treatment can inhibit at least 90% of plasma kallikrein activity within 30 minutes post-dosage amount, and in particular when the dosage amount of the compound of Formula A is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). The treatment can inhibit at least 95% of plasma kallikrein activity within 30 minutes post-dosage amount, and in particular when the dosage amount of the compound of Formula A (or a pharmaceutically acceptable salt and/or solvate thereof is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). Where the inhibition of plasma kallikrein activity is mentioned, inhibition of plasma kallikrein activity can be determined by time-dependent hydrolysis of fluorogenic substrate (e.g. (H-D-Pro-Phe-Arg-AFC; Peptide Protein Research) according to procedures known in the art. Inhibition of plasma kallikrein activity can be determined in plasma obtained from subjects that have taken a dosage amount of the compound of Formula A which has subsequently been activated with dextran sulfate to emulate a HAE situation.

A therapeutically effective concentration of the compound of Formula A can be achieved within 20 minutes post-dosage amount.

The Tₘₐₓ of the compound of Formula A can be between 30 minutes and 3 hours post-dosage amount, preferably between 30 minutes and 2 hours post-dosage amount.

The treatment can inhibit at least 90% of plasma kallikrein activity for at least the period of time between 45 minutes and 2 hours post-dosage amount, and in particular when the dosage amount of the compound of Formula A is between 100 mg and 200 mg (preferably 160 mg). The treatment can inhibit at least 90% of plasma kallikrein activity for at least the period of time between 20 minutes and 4 hours post-dosage amount, and in particular when the dosage amount of the compound of Formula A is between 100 mg and 200 mg (preferably 160 mg). The treatment can inhibit at least 90% of plasma kallikrein activity for at least the period of time between 30 minutes and 10 hours post-dosage amount, and in particular when the dosage amount of the compound of Formula A is between 300 mg and 800 mg (preferably 600 mg). The treatment can inhibit at least 95% of plasma kallikrein activity for at least the period of time between 20 minutes and 6 hours post-dosage amount, and in particular when the dosage amount of the compound of Formula A is between 300 mg and 800 mg (preferably 600 mg). The treatment can inhibit at least 99% of plasma kallikrein activity for at least the period of time between 20 minutes and 6 hours post-dosage amount, and in particular when the dosage amount of the compound of Formula A is between 300 mg and 800 mg (preferably 600 mg). Again, inhibition of plasma kallikrein activity can be determined in plasma obtained from subjects that have taken a dosage amount of the compound of Formula A which has subsequently been activated with dextran sulfate to emulate a HAE situation.

The pharmacodynamic effects of the compound of Formula A that are related to treating HAE can be maintained for at least 12 hours post-dosage amount, and in particular when the dosage amount of the compound of Formula A is between 300 mg and 800 mg (preferably 600 mg). The treatment can inhibit at least 50% of plasma kallikrein activity for at least 10 hours post-dosage amount, and in particular when the dosage amount of the compound of Formula A is between 100 mg and 200 mg (preferably 160 mg). Pharmacodynamic effects can mean at least (i) inhibition of plasma kallikrein, (ii) protection from HK cleavage/reduction of HK cleavage, (iii) protection from (or a reduction of) Factor XII cleavage to generate Factor XIIa, and/or (iv) protection from (or a reduction of) plasma prekallikrein cleavage to generate plasma kallikrein. Treatments according to the invention are therefore suitable candidates for being advantageously efficacious treatments of HAE attacks because they are fast-acting and potent (e.g. inhibitory) over a sufficiently long period of time.

As discussed above, in any of the treatments of the invention, the compound of Formula A can inhibit plasma kallikrein.

In any of the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the compound of Formula A can inhibit Factor XII cleavage to generate Factor XIIa. In any of the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the compound of Formula A can inhibit plasma prekallikrein cleavage into plasma kallikrein. In any of the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the compound of Formula A can result in the inhibition (e.g. blockage) of contact system activation for up to 6 hours post-dosage amount. In some embodiments, where a dosage amount of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg) is administered, the contact system activation can be inhibited (e.g. blocked) for at least 6 hours e.g. for between 6 hours and 12 or 18 hours post-dosage amount.

As demonstrated in the examples, the compound of Formula A is a potent inhibitor of plasma kallikrein. As already explained, inhibiting plasma kallikrein inhibits the cleavage of high molecular weight kininogen that contributes to an HAE attack. Additionally, and as demonstrated in Example 4, the compound of Formula A is also capable of reducing the cleavage of plasma prekallikrein and the generation of Factor XIIa (FXIIa) following activation of the contact system. These advantageous additional effects support the treatment of the invention being highly efficacious and are demonstrated in particular when the concentration of the compound of Formula A is at least 500 ng/mL of plasma. A plasma concentration of at least 500 ng/mL can be observed following administration of a dosage amount of at least about 60 mg (more specifically, at least about 70 or about 80 mg) of the compound of Formula A.

In the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), in addition to inhibiting plasma kallikrein, the treatments can also reduce the cleavage of plasma prekallikrein to generate plasma kallikrein and/or reduce the generation of Factor XIIa (FXIIa) following administration. Thus, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the treatments can block the cleavage of plasma prekallikrein to generate plasma kallikrein and/or block the cleavage of FXII to generate FXIIa.

### FIGURES

In the Figures, the term “Compound” means the compound of Formula A.

FIG. 1: X-ray powder diffraction pattern of the compound of Formula A as generated in Example 1.

FIG. 2A: Assay results showing plasma kallikrein inhibition activity of the compound of Formula A and a C1 inhibitor C1-INH in dextran sulfate (DXS)-activated diluted plasma.

FIG. 2B: Assay results showing plasma kallikrein inhibition activity of the compound of Formula A and a C1 inhibitor (C1-INH) in DXS-activated undiluted plasma.

FIG. 3A: Assay results comparing the plasma kallikrein inhibition activity of the compound of Formula A and C1-INH in DXS-activated diluted plasma.

FIG. 3B: Assay results comparing inhibition activity of the compound of Formula A and C1-INH following addition to pre-activated undiluted human plasma. Data are expressed as total fluorescence over time (Fluorescence Units) mean±SEM of n=3 experiments.

FIG. 4A: Assay (bioanalytical) results showing plasma concentrations of the compound of Formula A between 0 and 24 hours post-dose, in fasted subjects from eight (8) single ascending dose cohorts.

FIG. 4B: Table of Cₘₐₓ values determined from the assay (bioanalytical) results shown in FIG. 4A.

FIG. 5A: Assay results showing plasma kallikrein activity in DXS-activated undiluted plasma for cohorts 6 to 8 (160 mg, 300 mg, and 600 mg).

FIG. 5B: Assay results showing the mean plasma kallikrein activity and mean plasma concentration of the compound of Formula A in undiluted plasma in subjects from cohort 8 (600 mg dose).

FIG. 6A: Assay results showing the mean fluorescent kinetic measurements indicating a lag time in catalytic activity during contact system activation in DXS-activated undiluted plasma of a subject who has received a 600 mg dose of the compound of Formula A.

FIG. 6B: An enlargement of FIG. 6A between 0 and 5 mins following catalytic activation.

FIG. 7: Assay results showing mean percent HK protection at selected time points post-dosage in DXS-activated undiluted plasma for cohorts 6 to 8 (160 mg, 300 mg, and 600 mg), and a representation WES gel image of the immunoblot data.

FIG. 8: Assay results showing the effect of the compound of Formula A on DXS-activated HK cleavage at selected time points post-dosage in cohort 8 (600 mg), and a representation WES gel image of the immunoblot data.

FIG. 9: Assay results showing the effect of the compound of Formula A on DXS-activated plasma prekallikrein (PPK) cleavage, at selected time points post-dosage in cohort 8 (600 mg), and a representation WES gel image of the immunoblot data.

FIG. 10: Assay results showing the effect of the compound of Formula A on DXS-activated generation of FXIIa, at selected time points post-dosage in cohort 8 (600 mg), and a representation WES gel image of the immunoblot data.

FIG. 11: Assay (bioanalytical) results showing the effect of the plasma concentration of the compound of Formula A at various stages post-dose in cohort 8 (600 mg) at time points selected for HK, FXIIa, PPK analysis.

FIG. 12: Assay results showing no significant food effect on the plasma kallikrein inhibitory activity of the compound of Formula A in DXS-activated undiluted plasma.

FIGS. 13A and 13B: Assay results showing a time course of dextran sulfate-activated cleavage of HK in HAE whole undiluted plasma determined using western blotting, and a representative blot image.

FIGS. 14A and 14B: Assay results showing the dose response of the compound of Formula A on full length HK levels in dextran sulfate-activated healthy control plasma and HAE plasma, and representative WES system gel images.

FIG. 15a: Preliminary pharmacokinetic data from the phase 2 study.

FIG. 15b: PK profile of HAE patients from part 1 of the phase 2 study, overlaid with PK data from healthy volunteers.

FIG. 15c: Graph showing cumulative % of patients that used rescue medication within 12 hours of being administered the compound.

FIG. 15d: Graph showing cumulative % of patients with symptom relief (as assessed using the 7TQ method) within 12 hours of being administered the compound.

FIG. 15e: Graph showing comparison of time to symptom relief data for the compound with known clinical data for approved injectable product Ruconest®.

FIG. 15f: Graph showing cumulative % of patients with symptom relief (as assessed using the composite VAS score) within 12 hours of being administered the compound.

FIG. 15g: Graph showing mean composite VAS score within 12 hours of being administered the compound.

FIG. 15h: Graph showing comparison of mean composite VAS score for patients treated with the compound with known clinical data for approved injectable product Firazyr® (active ingredient, icatibant).

FIG. 15i: Graph showing the % of HAE attacks that did not worsen (as assessed using the 5LS method) within 12 hours of being administered the compound.

FIG. 16A: Mean plasma concentrations over time of 4 cohorts in the phase 1 multiple dose study.

FIG. 16B: Mean plasma concentrations over time (semi-logarithmic scale) of 4 cohorts in the phase 1 multiple dose study.

Embodiments provided herein may be more fully understood by reference to the following examples. These examples are meant to be illustrative of treatments provided herein, but are not in any way limiting. Indeed, the scope of the invention is defined by the claims.

While examples of certain particular embodiments are provided herein, it will be apparent to those skilled in the art that various changes and modifications may be made. Such modifications are also intended to fall within the scope of the appended claims.

### General Experimental Details

In the following examples, the following abbreviations and definitions are used:

| | |
|---|---|
| Aq | Aqueous solution |
| DCM | Dichloromethane |
| DMF | N,N-Dimethylformamide |
| DMSO | Dimethyl sulfoxide |
| DXS | Dextran sulfate |
| EtOAc | Ethyl Acetate |
| FXI | Factor XII |
| FXIIa | Factor XIIa |
| HK | High molecular weight kininogen |
| Hrs | Hours |
| HOBt | Hydroxybenzotriazole |
| IPA | 2-Propanol/Propan-2-ol/Iso-propanol |
| LCMS | Liquid chromatography mass spectrometry |
| Me | Methyl |
| MeCN | Acetonitrile |
| MeOH | Methanol |
| Min | Minutes |
| MS | Mass spectrum |
| NMR | Nuclear magnetic resonance |
| | spectrum-NMR spectra were recorded at a |
| | frequency of 400 MHz unless otherwise indicated |
| Pet. Ether | Petroleum ether fraction boiling at 60-80° C. |
| Ph | Phenyl |
| PKa | Plasma kallikrein |
| PPK | Plasma prekallikrein |
| SWFI | Sterile water for injection |
| Rt | room temperature |
| THF | Tetrahydrofuran |
| XRPD | X-ray powder diffraction |

All reactions were carried out under an atmosphere of nitrogen unless specified otherwise.

¹H NMR spectra were recorded on a Bruker (400 MHz) or on a JEOL (400 MHz) spectrometer with reference to deuterium solvent and at rt.

Molecular ions were obtained using LCMS which was carried out using a Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min, or using Agilent, X-Select, acidic, 5-95% MeCN/water over 4 min. Data was collected using a Thermofinnigan Surveyor MSQ mass spectrometer with electospray ionisation in conjunction with a Thermofinnigan Surveyor LC system.

Alternatively, molecular ions were obtained using LCMS which was carried out using an Agilent Poroshell 120 EC-C18 (2.7 μm, 3.0×50 mm) column with 0.1% v/v Formic acid in water [eluent A]; MeCN [eluent B]; Flow rate 0.8 mL/min and 1.5 minutes equilibration time between samples, gradient shown below. Mass detection was afforded with API 2000 mass spectrometer (electrospray).

Gradient:

| Time (min) | Eluent A (%) | Eluent B (%) |
|---|---|---|
| 0.00 | 95 | 5 |
| 0.20 | 95 | 5 |
| 2.00 | 5 | 95 |
| 3.00 | 5 | 95 |
| 3.25 | 95 | 5 |
| 3.50 | 95 | 5 |

Where products were purified by flash chromatography, ‘silica’ refers to silica gel for chromatography, 0.035 to 0.070 mm (220 to 440 mesh) (e.g. Merck silica gel 60), and an applied pressure of nitrogen up to 10 p.s.i accelerated column elution. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as the Autonom software provided as part of the ISIS Draw package from MDL Information Systems or the Chemaxon software provided as a component of MarvinSketch or as a component of the IDBS E-WorkBook.

X-Ray Powder Diffraction patterns were collected on a Philips X-Pert MPD diffractometer and analysed using the following experimental conditions (Method A), unless otherwise specified:

Tube anode: Cu

Generator tension: 40 kV

Tube current: 40 mA

Wavelength alpha1: 1.5406 Å

Wavelength alpha2: 1.5444 Å

Start angle [2θ]: 4

End angle [2θ]: 40

Continuous scan

Approximately 2 mg of sample under analysis was gently compressed on the XRPD zero back ground single obliquely cut silica sample holder. The sample was then loaded into the diffractometer for analysis.

### Example 1—Preparation of the Compound of Formula A

### A. 1-(4-Hydroxymethyl-benzyl)-1H-pyridin-2-one

4-(Chloromethyl)benzylalcohol (5.0 g, 31.93 mmol) was dissolved in acetone (150 mL). 2-hydroxypyridine (3.64 g, 38.3 mmol) and potassium carbonate (13.24 g, 95.78 mmol) were added and the reaction mixture was stirred at 50° C. for 3 hrs after which time the solvent was removed in vacuo and the residue taken up in chloroform (100 mL). This solution was washed with water (30 mL), brine (30 mL), dried (Na₂SO₄) and evaporated in vacuo. The residue was purified by flash chromatography (silica), eluent 3% MeOH/97% CHCl₃, to give a white solid identified as 1-(4-hydroxymethyl-benzyl)-1H-pyridin-2-one (5.30 g, 24.62 mmol, 77% yield).

[M+Na]⁺=238

### B. 1-(4-Chloromethyl-benzyl)-1H-pyridin-2-one

1-(4-Hydroxymethyl-benzyl)-1H-pyridin-2-one (8.45 g, 39.3 mmol), dry DCM (80 mL) and triethylamine (7.66 ml, 55.0 mmol) were cooled in an ice bath. Methanesulfonyl chloride (3.95 ml, 51.0 mmol) was added and stirred in ice bath for 15 min. The ice bath was removed and stirring continued at rt temperature overnight. The reaction mixture was partitioned between DCM (100 mL) and saturated aqueous NH₄C1 solution (100 mL). The aqueous layer was extracted with further DCM (2×50 mL) and the combined organics washed with brine (50 mL), dried over Na₂SO₄, filtered and concentrated to give 1-(4-chloromethyl-benzyl)-1H-pyridin-2-one (8.65 g, 36.6 mmol, 93% yield) as a pale yellow solid.

[MH]⁺=234.1

### C. Methyl 3-(methoxymethyl)-1-(4-((2-oxopyridin-1(2H)-yl)methyl)benzyl)-1H-pyrazole-4-carboxylate

Potassium carbonate (519 mg, 3.76 mmol) was added to a solution of methyl 3-(methoxymethyl)-1H-pyrazole-4-carboxylate (320 mg, 1.88 mmol; CAS no. 318496-66-1 (synthesised according to the method described in WO 2012/009009)) and 1-(4-(chloromethyl)benzyl)pyridin-2(1H)-one (527 mg, 2.26 mmol) in DMF (5 mL) and heated at 60° C. overnight. The reaction mixture was diluted with EtOAc (50 mL) and washed with brine (2×100 mL), dried over magnesium sulfate, filtered and reduced in vacuo. The crude product was purified by flash chromatography (40 g column, 0-100% EtOAc in isohexanes) to afford two regioisomers. The second isomer off the column was collected to afford methyl 3-(methoxymethyl)-1-(4-((2-oxopyridin-1(2H)-yl)methyl)benzyl)-1H-pyrazole-4-carboxylate (378 mg, 1.01 mmol, 53.7% yield) as a colourless gum.

[MH]⁺=368.2

### D. 3-(Methoxymethyl)-1-(4-((2-oxopyridin-1(2H)-yl)methyl)benzyl)-1H-pyrazole-4-carboxylic acid

To methyl 3-(methoxymethyl)-1-(4-((2-oxopyridin-1(2H)-yl)methyl)benzyl)-1H-pyrazole-4-carboxylate (3.77 g, 10.26 mmol) in THF (5 mL) and MeOH (5 mL) was added 2M NaOH solution (15.39 ml, 30.8 mmol) and stirred at rt overnight. 1M HCl (50 mL) was added and extracted with EtOAc (50 mL). The organic layer was washed with brine (50 mL), dried over magnesium sulfate, filtered and reduced in vacuo to give 3-(methoxymethyl)-1-(4-((2-oxopyridin-1(2H)-yl)methyl)benzyl)-1H-pyrazole-4-carboxylic acid (1.22 g, 3.45 mmol, 33.6% yield) as a white powder.

[MH]⁺=354.2

### E. 3-Fluoro-4-methoxy-pyridine-2-carbonitrile

To a large microwave vial, copper (1) cyanide (1.304 g, 14.56 mmol) was added to a solution of 2-bromo-3-fluoro-4-methoxypyridine (1 g, 4.85 mmol) in DMF (5 mL). The reaction vial was sealed and heated to 100° C. for 16 hrs. The reaction mixture was diluted with water (20 mL) and EtOAc (20 mL). The thick suspension was sonicated and required additional water (40 mL) and EtOAc (2×50 mL) with sonication to break-up the solid precipitated. The combined layers were filtered through a plug of celite and the organic layer isolated, washed with brine (50 mL), dried over magnesium sulfate, filtered and the solvent removed under reduced pressure to give a pale green solid identified as the desired compound 3-fluoro-4-methoxy-pyridine-2-carbonitrile (100 mg, 0.578 mmol, 12% yield)

### F. (3-Fluoro-4-methoxy-pyridin-2-ylmethyl)-carbamic acid tert-butyl ester

3-Fluoro-4-methoxy-pyridine-2-carbonitrile (100 mg, 0.578 mmol) was dissolved in anhydrous methanol (10 mL, 247 mmol) and nickel chloride hexahydrate (14 mg, 0.058 mmol) was added followed by di-tert-butyl dicarbonate (255 mg, 1.157 mmol). The resulting pale green solution was cooled in an ice-salt bath to −5° C. and then sodium borohydride (153 mg, 4.05 mmol) was added portionwise maintaining the reaction temperature ˜0° C. The deep brown solution was left to stir at 0° C. and slowly allowed to warm to rt and then left to stir at rt for 3 hrs. The reaction mixture was evaporated to dryness at 40° C. to afford a black residue which was diluted with DCM (10 mL) and washed with sodium hydrogen carbonate (10 mL). An emulsion formed so the organics were separated via a phase separating cartridge and concentrated. The crude liquid was purified by chromatography eluting with EtOAc/iso-Hexane to afford the title compound, (3-fluoro-4-methoxy-pyridin-2-ylmethyl)-carbamic acid tert-butyl ester as a clear yellow oil (108 mg, 62% yield)

[MH]⁺=257

### G. C-(3-Fluoro-4-methoxy-pyridin-2-yl)-methylamine hydrochloride salt

(3-Fluoro-4-methoxy-pyridin-2-ylmethyl)-carbamic acid tert-butyl ester (108 mg, 0.358 mmol) was taken up in iso-propyl alcohol (1 mL) and then HCl (6N in iso-propyl alcohol) (1 mL, 0.578 mmol) was added at rt and left to stir at 40° C. for 2 hrs. The reaction mixture was concentrated under reduced pressure and then triturated with ether, sonicated and then decanted to give a cream coloured solid (75 mg, 55% yield) identified as C-(3-fluoro-4-methoxy-pyridin-2-yl)-methylamine hydrochloride salt.

[MH]⁺=157

### Example 1a—N-[(3-Fluoro-4-methoxypyridin-2-yl)methyl]-3-(methoxymethyl)-1-({4-[(2-oxopyridin-1-yl)methyl]phenyl}methyl)pyrazole-4-carboxamide (Compound of Formula A)

3-(Methoxymethyl)-1-(4-((2-oxopyridin-1(2H)-yl)methyl)benzyl)-1H-pyrazole-4-carboxylic acid (825 mg, 2.34 mmol) and C-(3-fluoro-4-methoxy-pyridin-2-yl)-methylamine hydrochloride salt (450 mg, 2.34 mmol) were dissolved in DCM while cooling to 0° C. 1-Ethyl-3-(3-dimethylaminopropyl)carbodiimide hydrochloride (627.0 mg, 3.27 mmol), HOBt (378.8 mg, 2.80 mmol) and triethylamine (1.63 mL, 1182 mmol) were added while stirring, the mixture allowed to warm to rt and stirring continued for 20 hrs. Chloroform (50 mL) was added, the mixture was washed with saturated NaHCO₃(aq) and reduced in vacuo. The crude material was purified by chromatography eluting with methanol/DCM. The solvent was removed in vacuo and the resulting solid triturated with diethyl ether. The resulting solids were collected by filtration to afford the compound of Formula A.

[MH]⁺=492.0

NMR (CD₃OD) δ: 3.41 (31H, s), 4.03 (31H, s), 4.65 (2H, s), 4.72 (21H, d, J=2.3 Hz), 5.24 (2H, s), 5.37 (2H, s), 6.44 (1H, td, J=1.4, 6.8 Hz), 6.62 (1H, d, J=9.0 Hz), 7.18-7.22 (1H, m), 7.31-7.38 (4H, m), 7.56-7.60 (1H, m), 7.75 (1H, dd, J=1.9, 7.1 Hz), 8.18 (1H, s), 8.27 (1H, d, J=5.6 Hz) ppm.

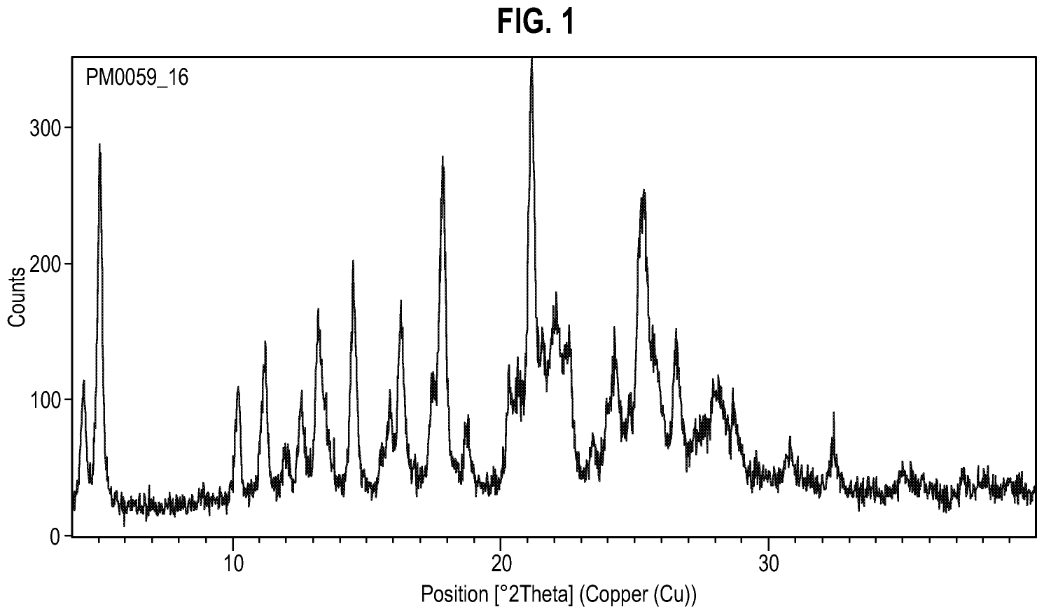

An XRPD diffractogram of the compound of Formula A resultant from the above procedure is shown in FIG. 1.

Peak position table:

| No. | Pos. [°2Th.] | Rel. Int. [%] |
|---|---|---|
| 1 | 4.436 | 32.36 |
| 2 | 5.0471 | 58.74 |
| 3 | 10.2255 | 43.07 |
| 4 | 11.2061 | 48.44 |
| 5 | 12.0101 | 16.4 |
| 6 | 12.5494 | 37.17 |
| 7 | 13.165 | 67.26 |
| 8 | 14.4984 | 38.94 |
| 9 | 15.8919 | 23.54 |
| 10 | 16.2983 | 34.56 |
| 11 | 17.4492 | 36.63 |
| 12 | 17.8564 | 71.49 |
| 13 | 18.6888 | 21.9 |
| 14 | 20.285 | 26.12 |
| 15 | 21.1598 | 100 |
| 16 | 22.04 | 87.76 |
| 17 | 22.5857 | 36.38 |
| 18 | 23.4408 | 14.33 |
| 19 | 24.3045 | 31.11 |
| 20 | 25.1655 | 78.97 |
| 21 | 25.3728 | 93.91 |
| 22 | 26.4946 | 56.79 |
| 23 | 27.991 | 76.91 |
| 24 | 28.7495 | 22.99 |
| 25 | 30.7611 | 13.4 |
| 26 | 32.413 | 17.2 |
| 27 | 37.2144 | 14.13 |
| 28 | 38.1171 | 14.14 |

### Example 2—Preparation of a Dosage Form Comprising the Compound of Formula A

Blending and Roller Compaction

Equipment: Freund Vector TFC Lab Micro Roller Compactor and Granulator (the roller compactor and granulator are separate entities). The equipment parameters are below:

| Parameter | Range Used |
|---|---|
| Screw Speed (rpm) | 10.0-20.0 |
| Roll Speed (rpm) | 1.0-2.0 |
| Roll Force (kN) | 0.50-12.00 |
| Granulator Screen Size (mm) | 1 |

Method

Two tablet formulations (Tablets A and B) were prepared according to the following method at 30 g blend scale to produce tablets having components in the amounts shown below.

| | | wt % (compared to the | |
|---|---|---|---|
| | | total mass of the tablet) | |
| | Component | Tablet A | Tablet B |
| Intragranular | The compound of | 42.5 | 47.5 |
| | Formula A | | |
| | Microcrystalline | 34.5 | 29.5 |
| | Cellulose | | |
| | (Avicel PH101) | | |
| | Croscarmellose | 4 | 4 |
| | Sodium (Ac-di-sol) | | |
| | Polyvinyl | 3 | 3 |
| | Pyrrolidone | | |
| | (Kollidon 30) | | |
| Extragranular | Microcrystalline | 15 | 15 |
| | Cellulose | | |
| | (Avicel PH102) | | |
| | Magnesium | 1 | 1 |
| | Stearate | | |

For each of the tablets, blends were prepared by passing the intragranular components through a 355 μm sieve at a suitable scale for the scope of the roller compactor in a glass vessel using a Turbula Blender at 34 rpm. The blend was then run through the roller compactor using the parameters described above. The ribbons produced were collected into a suitably sized container. The collected ribbons were then subjected to the granulator fixed with a 1 mm screen and the resultant granules were collected for further downstream processing.

Tabletting

Equipment: RIVA Mini single station Tablet Press. The equipment parameters are shown below:

| Parameter | Range Used |
|---|---|
| Tooling Diameter | 8 mm round |
| Force (kN) | 6-10 |
| Fill Weight (mg) | 178-300 |

The granules were subsequently blended with their extragranular excipients, respectively. The extragranular excipients were prepared by screening through a 355 μm sieve in a glass vessel using a Turbula Blender at 34 rpm. The target tablet weight was then dispensed and manually compressed into tablets. Tablet A was compressed at 7.2 to 8.8 kN compression force. Tablet B was compressed at 6.9 to 7.7 kN compression force.

The tablets were found to be robust. Tablets A and B were subsequently submitted for long-term stability testing.

The production of tablets per the method described above has been scaled to 180 g with a roller compaction time of approximately 60 minutes.

### Example 3—Comparison of the Compound of Formula a with a C1 Inhibitor (C1-INH)

Aim: To identify the biochemical and biophysical properties of the compound of Formula A that contribute to its optimal efficacy in controlling the Kallikrein Kinin System in plasma. These properties are then compared to C1-INH as a therapeutic benchmark for HAE.

Methods:

Plasma kallikrein inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human plasma kallikrein (Protogen) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC₅₀ value for the test compound was determined.

The rate of formation of the enzyme-inhibitor complex (Kₒₙ) was determined using purified PKa rapidly mixed with a solution containing fluorogenic substrate and a concentration range of inhibitor. The time-dependent establishment of inhibition was then used to calculate the rate of formation of the enzyme-inhibitor complex for each concentration of inhibitor. The Kₒₙ was calculated by plotting the rate of inhibition versus the inhibitor concentration. Data in Table 1 are presented in μM⁻¹ sec⁻¹.

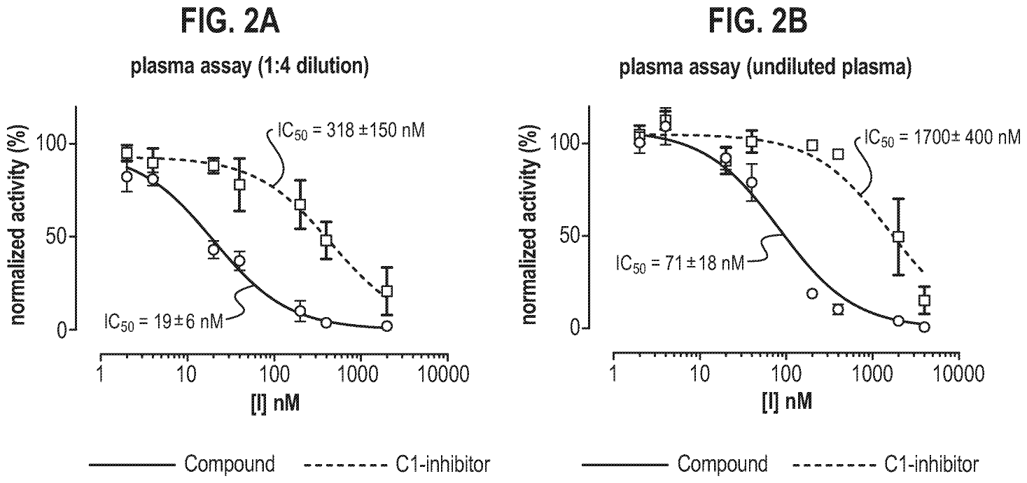

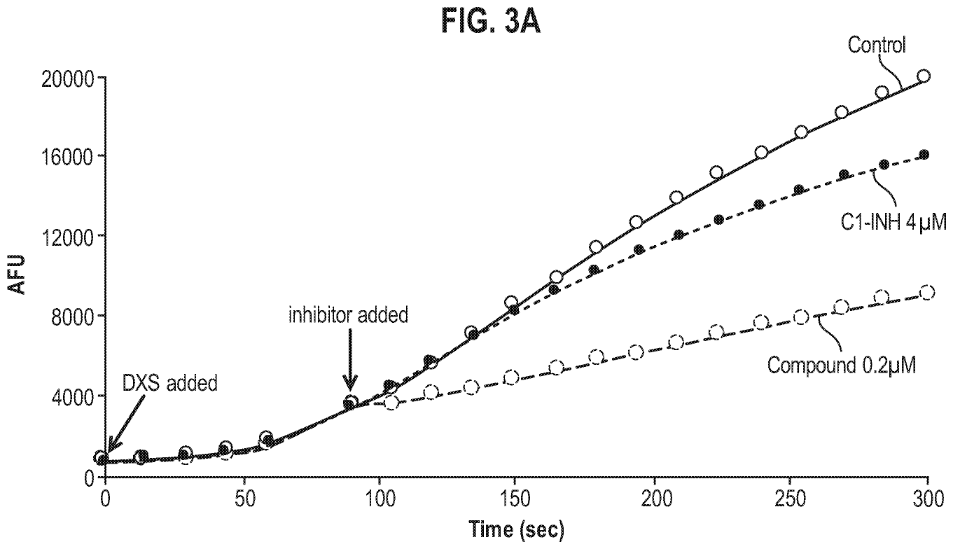

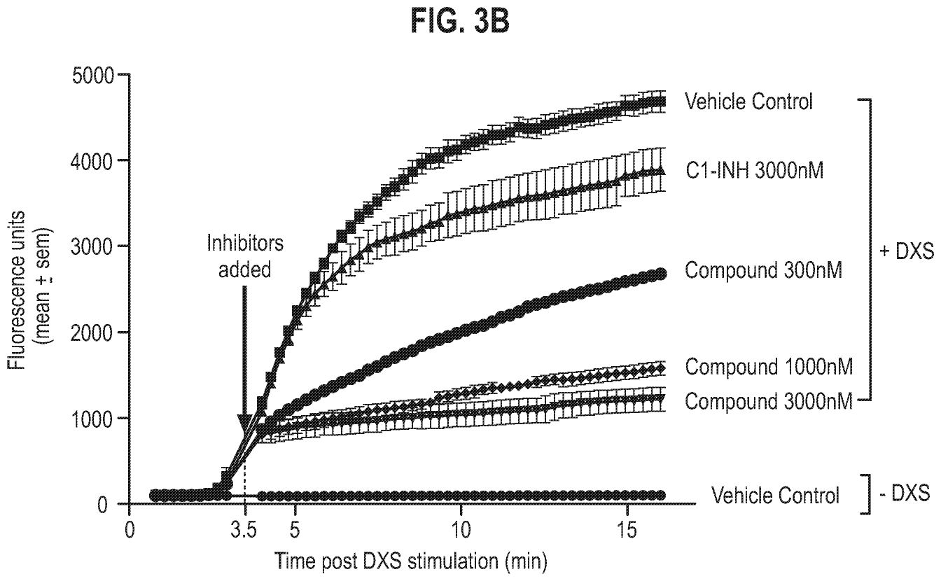

Catalytic activity of PKa in dextran sulfate-activated (DXS, Sigma; 10 μg/ml) plasma (1:4 diluted or undiluted, VisuCon-F control plasma, Affinity Biologicals Inc) was determined by the time-dependent hydrolysis of fluorogenic substrate. For IC₅₀ and efficacy determination, the compound of Formula A or C1-INH (Sigma Cat #E0518) were added either before (FIGS. 2A and 2B) or after (FIG. 3A) the addition of DXS to the plasma.

DXS-activated cleavage of HK in undiluted plasma was performed in the absence or presence of 300 nM PKa inhibitor and quantified by SDS-PAGE gel electrophoresis, using 7.5% Criterion TGX Precast gels (Biorad). Transfer was made onto Immunobilon-FL PVDF membrane. Image analysis was done using the LICOR imaging system. Mouse monoclonal anti-HK antibody (MAB15692, R&D systems) was used for traditional immunoblotting. Data presented as % of HK remaining after 20 min incubation with DXS compared to HK levels in unactivated plasma (Table 1).

Plasma free fraction was determined using “Rapid Equilibrium Dialysis” system (Thermo Scientific), test compounds were prepared at 5 μM in neat human plasma and dialysed against phosphate buffer for 5 hrs at 37° C. Quantification of the compound partitioned in two chambers of the dialysis device was performed via LCMS/MS. Fraction of compound unbound to plasma proteins presented as % of total.

The ability of the compound to inhibit the enzyme activity of pre-activated plasma was assessed by addition of the compound after DXS stimulation. Aliquots of plasma (20 μL) were mixed with a 2.5 μL solution containing 1,300 mM fluorogenic substrate (H-DPro-Phe-Arg-AFC) and a 2.5 μL solution of dextran sulphate (DXS; 100 μg/mL) which acted as an activator of the plasma kallikrein-kinin pathway. Enzyme activity was immediately measured by monitoring the accumulation of fluorescence liberated from the substrate by substrate cleavage over 16 minutes. At 3.5 minutes post DXS addition 5 μl of inhibitors or water control are were added into each well. The compound was tested at concentrations of 300, 1000 and 3000 nM. C1-INH at a concentration of 3000 nM and vehicle controls were also included. Data are presented in FIG. 3B.

Results:

As shown in FIG. 2, in assays using the fluorogenic substrate, the compound of Formula A appears to be a highly potent inhibitor of PKa with 17-fold and 20-fold potency vs. exogenously added C1-INH in diluted plasma (FIG. 2A) and undiluted plasma (FIG. 2B), respectively.

Table 1 showing the biochemical profile of the therapies tested in this example.

| | IC₅₀ | IC₅₀ | | Plasma | |
|---|---|---|---|---|---|
| | purified | plasma | Kₒₙ | Free | HK |
| | enzyme | enzyme | (μM⁻¹ | Fraction | protection |
| | nM | nM | sec⁻¹) | % | % |
| C1-INH | 50 | 1700 | 0.04 | | |
| Compound of | 6 | 71 | 11 | 25 | 85 |
| Formula A | | | | | |

FIG. 3A shows a comparison of the effects of the two inhibitors: compound of Formula A and C1-INH, on plasma kallikrein activity in plasma (diluted 1:4) activated with DXS. Both inhibitors were added at concentrations ten times their IC₅₀ to plasma approximately 100 seconds after the addition of the DXS.

FIG. 3B shows that addition of the compound of Formula A after the activation of plasma causes rapid and dose dependent inhibition of enzyme activity compared to the slower action of C1-INH.

Table 2 shows the biochemical potency and selectivity of the compound of Formula A against human isolated enzymes using literature methods as for the above-described in vitro plasma kallikrein assay.

| The compound | Ki (nM) |
|---|---|
| Plasma Kallikrein | 3.02 |
| Selectivity vs PKa | Fold |
| Tissue Kallikrein (KLK1) | >6000 |
| Factor XIa | >6000 |
| Factor XIIa | >6000 |
| Thrombin | >6000 |
| Trypsin | >6000 |

### Example 4—Phase I Single Ascending Dose Study in Healthy Males and Food Effect of the Compound

Aim: To evaluate the pharmacodynamic (PD) effects of the compound of Formula A when orally administered using ex vivo whole plasma assays for plasma kallikrein catalytic activity and HK cleavage, in samples from a Phase 1 Single Ascending Dose Study in healthy adult males. Also, an aim was to investigate safety, tolerability and pharmacokinetic (PK) effects of the compound of Formula A when orally administered.

Methods:

This study was a randomized, double-blind, placebo-controlled single ascending dose (SAD) and crossover studies for food effect and capsule/tablet formulations.

64 healthy male participants (n=6 active, 2 placebo per cohort, 8 SAD cohorts) were administered single ascending doses of the compound of Formula A: 5, 10, 20, 40, 80, 160, 300 or 600 mg in a capsule.

8 participants were administered 100 mg the compound of Formula A in a crossover study of the capsule and a tablet formulation.

12 participants were administered 600 mg the compound of Formula A in a food effect crossover study.

Samples for pharmacokinetic (PK) and PD assessment were taken at repeated intervals over 48 hours.

Plasma samples used for PK assessment were analysed using a validated liquid chromatography tandem mass spectrometry (LC MS/MS) method.

PD measurements were determined in dextran sulfate (DXS) stimulated undiluted plasma using a fluorogenic enzyme assay and capillary based HK cleavage immunoassay.

Catalytic activity of PKa in DXS-stimulated (Sigma; 10 μg/mL) plasma samples from the compound of Formula A phase 1 study was determined by the time-dependent hydrolysis of fluorogenic substrate in all samples from all parts of the study.

The time until appearance of detectable amidolytic enzyme activity in DXS-stimulated plasma (lag time) was calculated from the catalytic activity assay. The detection sensitivity of the rate of catalytic activity in plasma based on using a Spark (Tecan) fluorimeter is a fluorescence increase to reach 1ΔF unit/sec.

DXS-stimulated cleavage of HK, in undiluted plasma was quantified by capillary-based immunoassay on the Wes System (ProteinSimple) using monoclonal anti-HK antibody and chemiluminescence-based detection. Plasma kallikrein mediated HK cleavage in undiluted citrated human plasma was induced by contact system activation with DXS (6.25 μg/ml) at 4° C. in selected samples from the SAD phase.

DXS-stimulated cleavage of plasma prekallikrein and Factor XII (FXII) were quantified by capillary-based immunoassay on the Wes System (ProteinSimple) analogously.

Results:

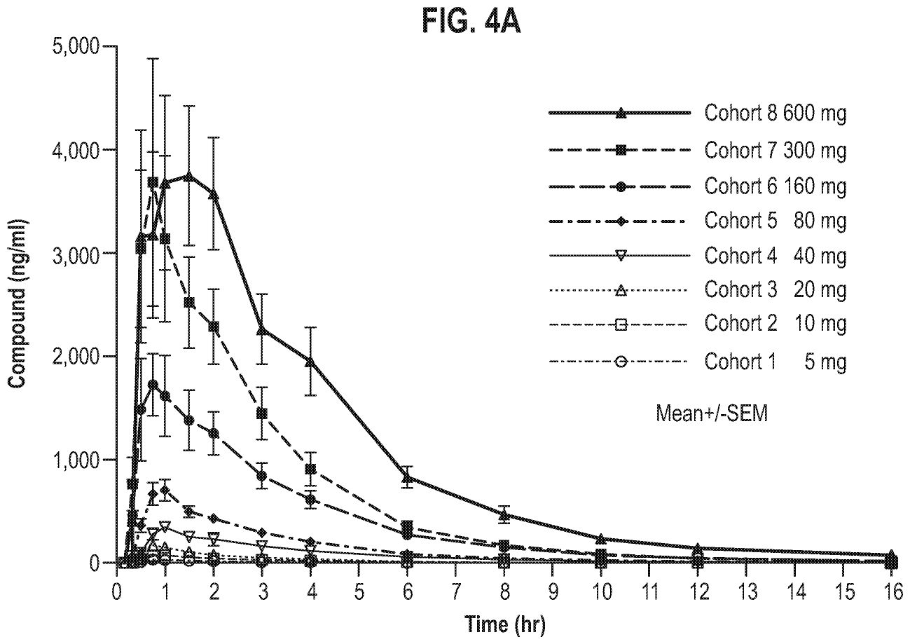

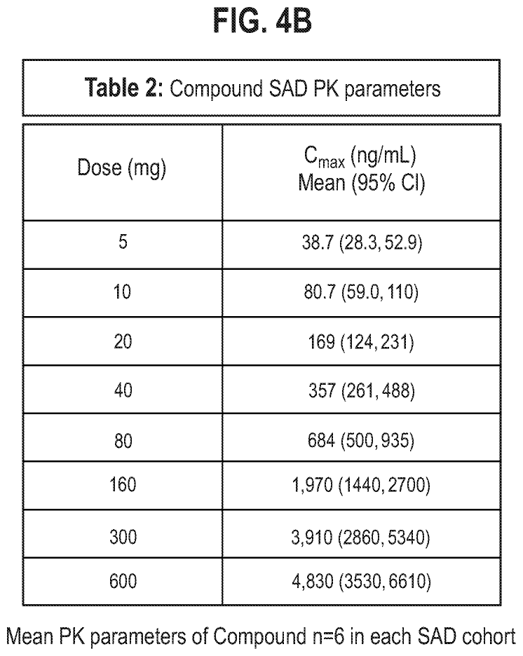

FIG. 4A shows the plasma concentrations of the compound of Formula A from 0 to 24 hours post-dose. As can be seen, when orally administered, the compound of Formula A achieved rapid and dose-dependent plasma exposure over the range of doses tested from 5 mg to 600 mg. FIG. 4A shows the concentration curves and FIG. 4B shows the Cₘₐₓ for each SAD cohort. The compound of Formula A was administered as a capsule formulation and the subject was in the fasted state.

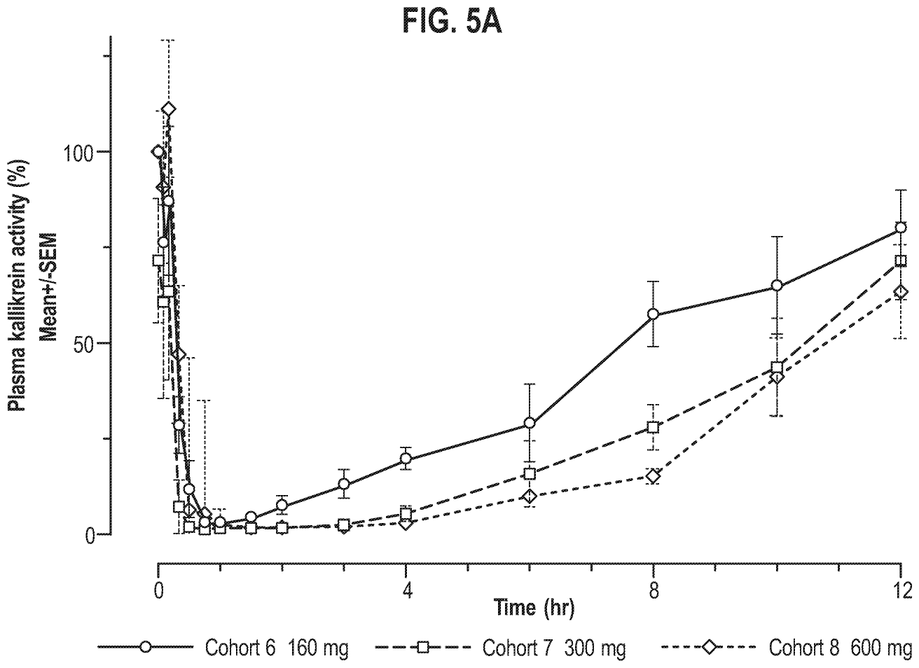

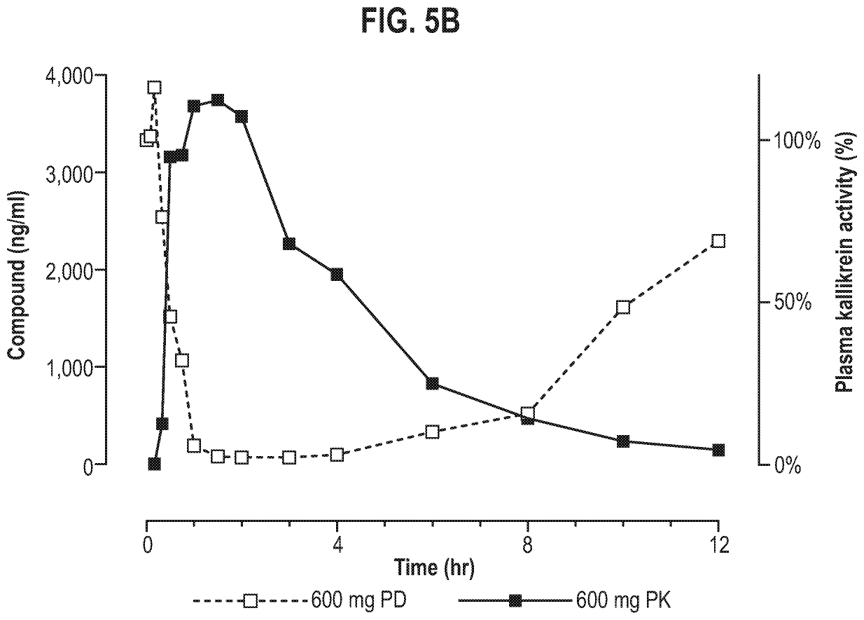

FIG. 5A shows enzyme assays in activated undiluted plasma performed on samples from cohorts 6, 7, and 8. Doses 160 mg and above demonstrated >90% average inhibition of plasma kallikrein catalytic activity between 45 min and 2 hr for cohort 6, between 20 min and 4 hr for cohort 7. A 600 mg dose (cohort 8) provided >90% inhibition of plasma kallikrein catalytic activity between 30 min and 6 hr post-dose and >50% inhibition for 10 hr (FIG. 5B).

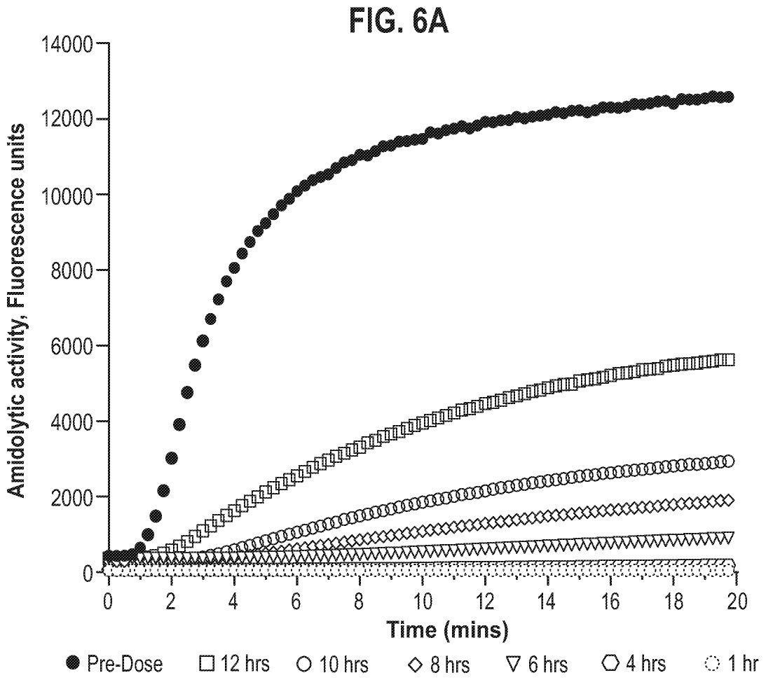

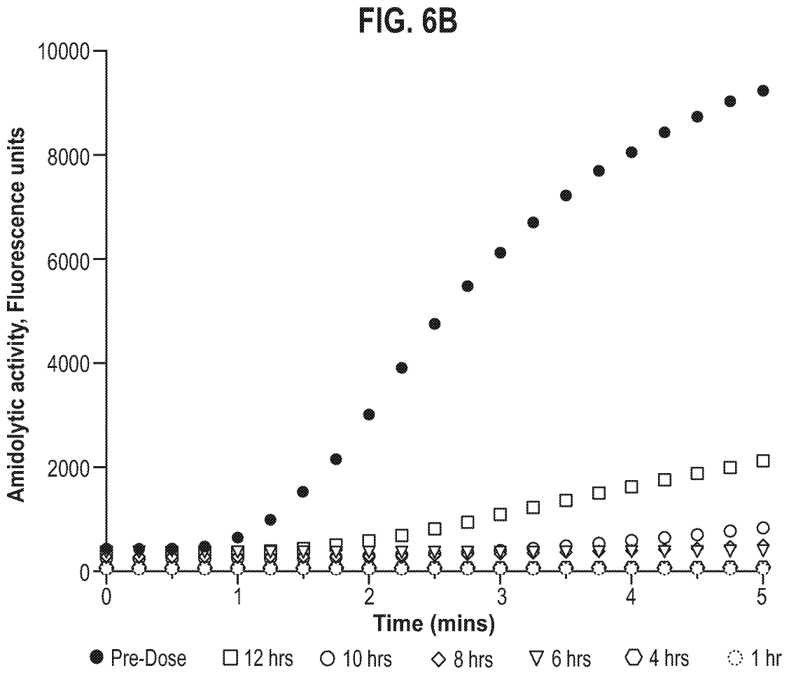

The kinetic fluorescent measurements from the undiluted plasma enzyme assay can be plotted as assay progression curves (FIGS. 6A and 6B). These curves highlight that the compound of Formula A not only has an inhibitory effect on enzyme activity but also increases the time until appearance of catalytic activity during contact system activation (lag time). At early time points post-dose administration, plasma samples did not display detectable catalytic activity even after prolonged activation with the potent activator DXS. In this test, the subject was administered with 600 mg dose in a tablet formulation.

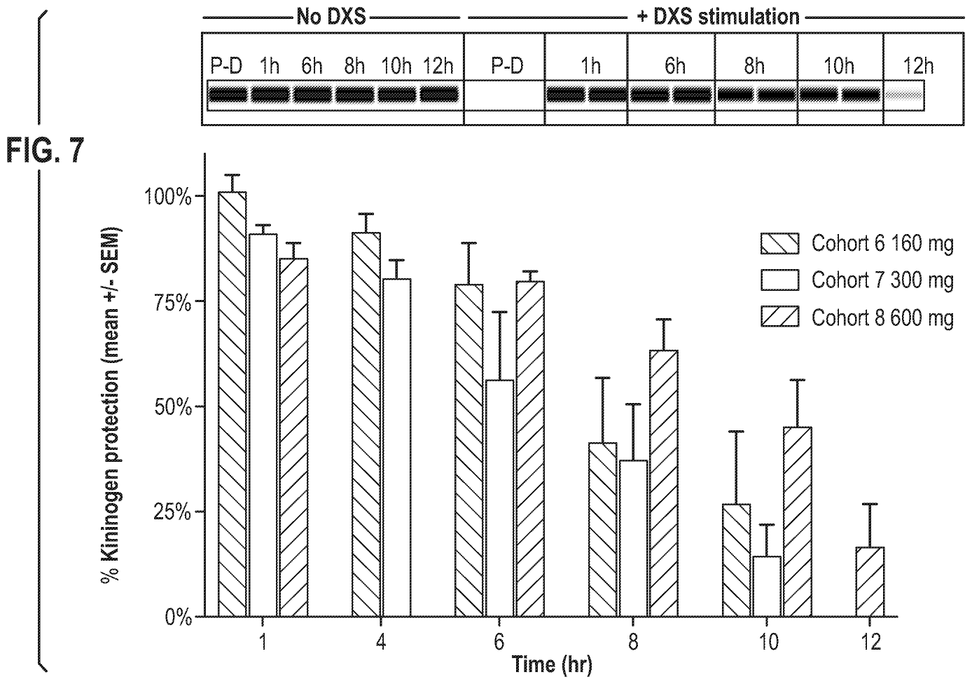

FIG. 7 shows the mean percent HK protection in DXS-activated undiluted plasma (SAD cohort 6 (160 mg), 7 (300 mg) and 8 (600 mg)). As shown, all three doses of the compound of Formula A were able to inhibit plasma kallikrein catalytic activity above 90% for a period of time. The duration of these PD effects was dose proportional. The compound of Formula A is shown to protect HK from DXS-activated cleavage in undiluted plasma for at least 10 hr following a single 600 mg dose.

In FIG. 7, the representative WES system gel image was generated in duplicate undiluted plasma samples+/−DXS activation from a single subject in cohort 8 who received 600 mg of the compound of Formula A compared with pre-dose (P-D).

In FIG. 7, HK cleavage was evaluated following DXS activation of undiluted plasma samples at selected time points from cohorts 6 to 8. Data are expressed as Mean+/−SEM, n=6.

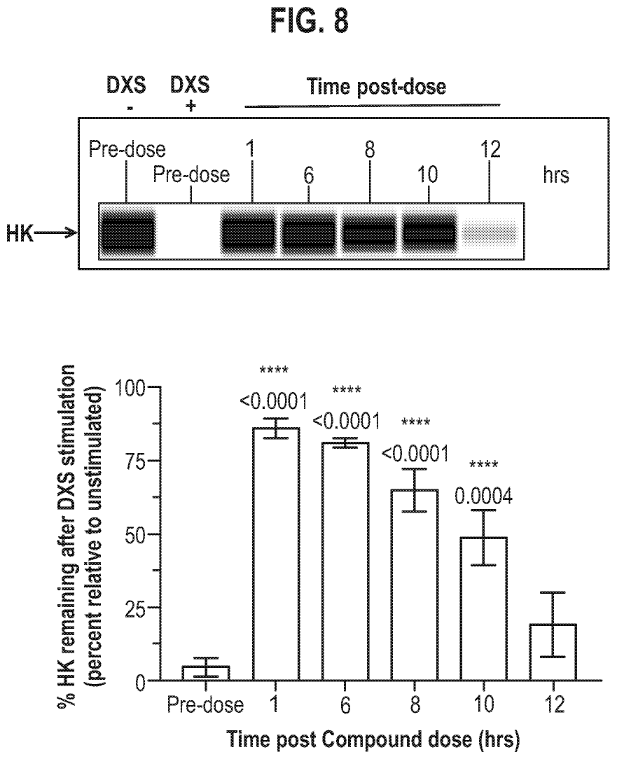

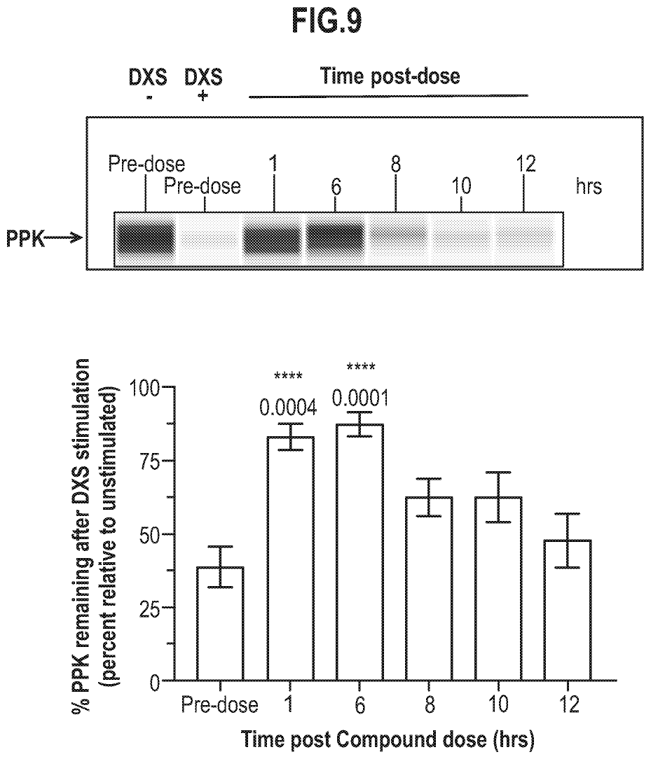

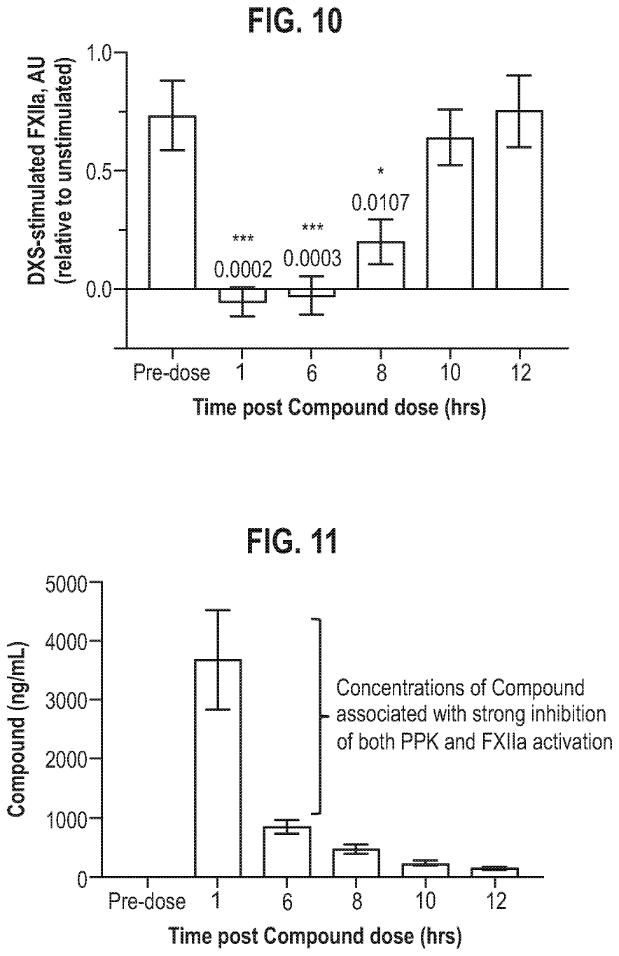

To assess whether the compound of Formula A also reduced the generation of plasma kallikrein and Factor XIIa, immunoassays were used to quantify levels of contact system proteins in DXS-activated plasma at pre-dose and up to 12 hrs post-dose of 600 mg orally administered in capsules. The results from these assays are shown in FIGS. 8 to 11 and shows that the compound of Formula A not only reduces HK cleavage but also reduces PPK cleavage and reduces the generation of FXIIa. These results suggest that the compound of Formula A inhibits the contact activation system via interruption of the positive feedback loop mediated by PKa stimulated activation of FXII.

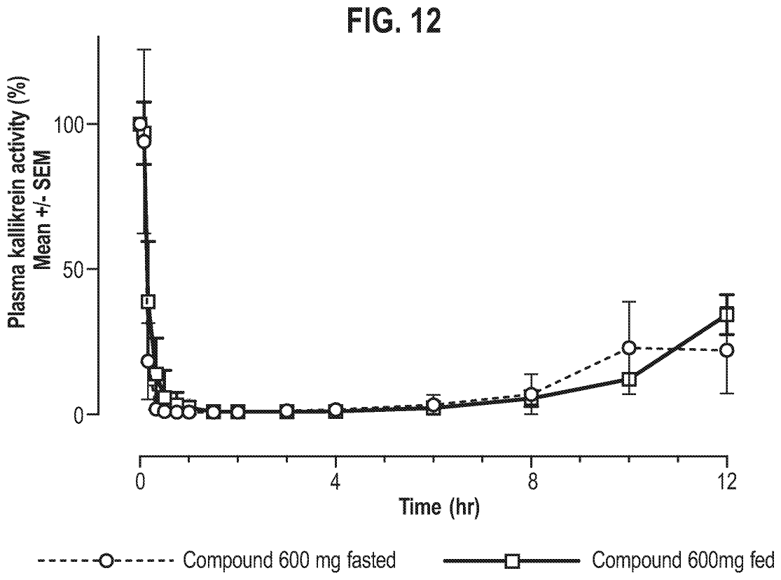

FIG. 12 shows that no significant food-effect was observed on the pharmacodynamic (PD) profile of a 600 mg tablet provided in fed and fasted state. As can be seen, the PD effects are rapidly observed in fed and fasted state with plasma kallikrein inhibition of >90% achieved by 30 minutes in both states.

No serious adverse events were reported in the phase I trial. There were also no tolerability signals. No subjects withdrew from the trial.

These data demonstrate that the compound of Formula A has an inhibitory effect on the bradykinin and contact activation systems. As discussed above, these pharmacodynamic effects are implicated in disorders such as HAE. These data also show that the compound of Formula A has a pharmacokinetic profile suitable for oral administration.

### Example 5—Immunoassays Investigating the Compound of Formula a in the Protection of High Molecular Weight Kininogen (HK) from PKa-Mediated Cleavage in HAE and Control Plasma

Method:

High molecular weight kininogen (HK) cleavage in undiluted citrated human plasma was induced by contact system activation with dextran sulfate (DXS, Sigma #31395-10G; 6.25 μg/ml) on wet ice. Pooled normal (CONTROL) human plasma (VisuCon-F Frozen Normal Control plasma) was purchased from Affinity Biologicals Inc. A working stock of 10 mM of the compound of formula A (“the compound”) in DMSO was prepared and diluted in 1×PBS to the respective final concentrations described. HAE plasma was obtained from HAE subjects (n=6) and C1-inhibitor deficiency was confirmed by western blotting. Protection of HK from PKa mediated cleavage in DXS-stimulated whole undiluted plasma was then determined by two methods; traditional western blotting and a semi-automated capillary-based immunoassay.

Western blotting: SDS-PAGE gel electrophoresis was done using 7.5% Criterion TGX Precast gels (Bio-rad). Transfer was made onto Immobilon-FL PVDF membrane. Image analysis was performed using the LICOR imaging system. Mouse monoclonal anti-human HK antibody (MAB15692, R&D systems) was used for traditional immunoblotting.

Capillary-Based Immunoassay on the WES System (ProteinSimple):

Preparation of samples: Combine one part 5× fluorescent master mix with four parts of the 1:200 plasma sample. Vortex to mix. Heat the samples+fluorescent master mix and the biotinylated ladder at 95° C. for 5 minutes, vortex, and load onto the WES plate. Monoclonal anti-human HK antibody was used for this chemiluminescence-based detection method using the Wes System (ProteinSimple). Analysis: Collect the peak area measurement obtained in the Compass software (cbz file) for the full-length HK molecular weight of the respective time-point sample with DXS-induced activation. The peak area is defined as the area calculated for the spectral peak profile for HK. To measure the plasma kallikrein inhibition by the compound, the percent full-length HK detected was calculated.

Results:

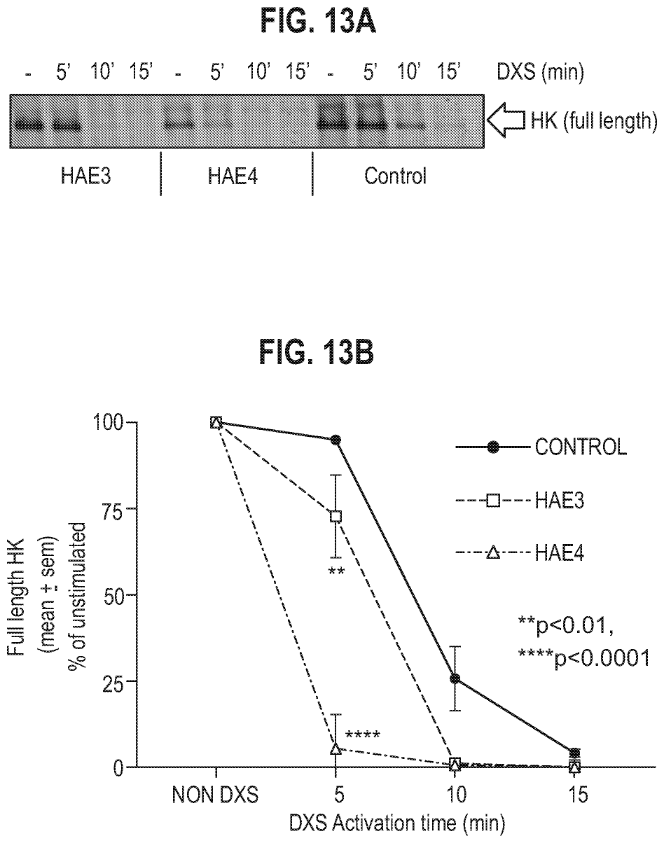

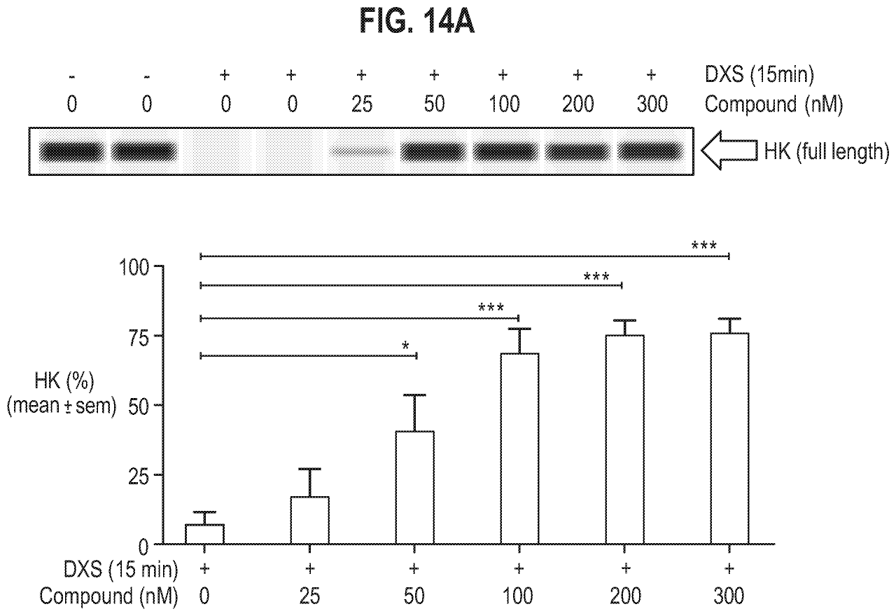

FIGS. 13A and 13B show the time course of dextran sulfate-activated cleavage of HK in HAE whole undiluted plasma determined using western blotting, and a representative blot.

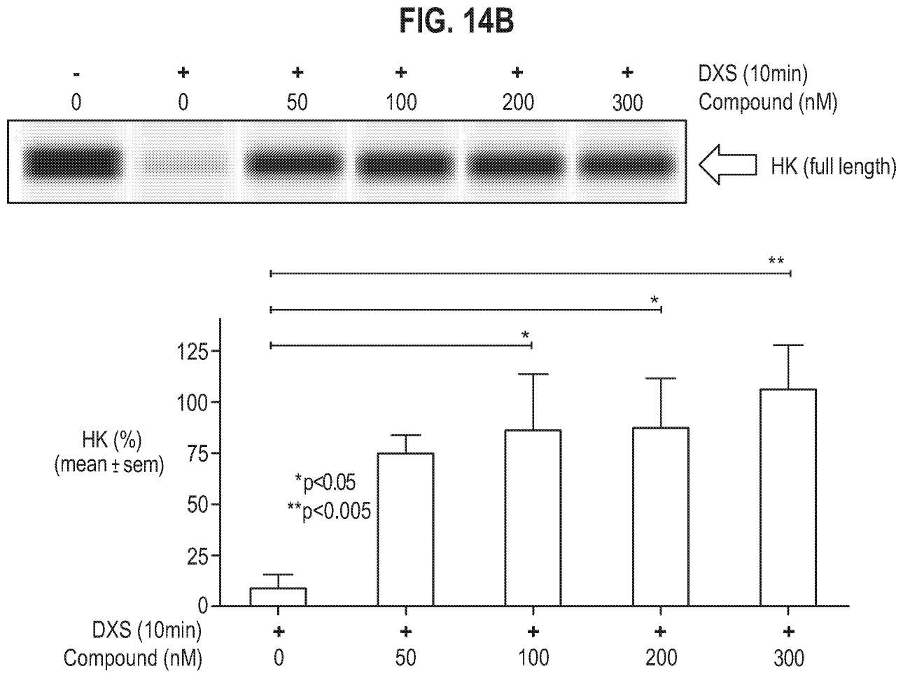

FIGS. 14A and 14B shows a representative WES system gel image and that the compound of Formula A provides dose dependent protection against HK cleavage in both HAE and healthy control plasma stimulated with dextran sulfate determined by capillary-based immunoassay using the WES system.

### Example 6—Phase 2 Study of the Compound of Formula A

Aim: To evaluate the efficacy and safety of the compound of Formula A in the on-demand treatment of angioedema attacks in adult subjects with hereditary angioedema type I or II.

Methods:

The study was a randomized, double-blind, placebo-controlled, phase 2, cross-over clinical trial evaluating the efficacy and safety of the compound of formula A (“the compound”), an oral plasma kallikrein inhibitor, in the on-demand treatment of angioedema attacks in adult subjects with hereditary angioedema type I or II (EudraCT number: 2018-004489-32).

Objectives:

Primary Objective:
To investigate the efficacy of the compound compared to placebo in halting the progression of a peripheral or abdominal attack of hereditary angioedema (HAE).

Secondary Objectives:
To investigate the safety and tolerability of the compound.
To investigate the pharmacokinetic (PK) profile of the compound when taken during the intercritical period between HAE attacks.
To investigate the pharmacodynamic (PD) profile of the compound in reducing the concentration of residual cleaved high molecular weight kininogen (HK) during the intercritical period between HAE attacks.
To investigate the PD profile of the compound in reducing activated plasma enzyme activity during the intercritical period between HAE attacks.

Setup:

This was a phase 2, two-part, two-sequence, two-period (2×2) cross-over clinical trial. Subjects with HAE type I or II were recruited through HAE treatment centres in Europe and US.

In Part 1, subjects received a single oral dose of 600 mg of the compound to investigate the safety, PK and PD of the compound during the intercritical period between HAE attacks.

Eligible adult subjects ≥18 years old underwent a screening assessment for study inclusion and to receive study drug, followed by a 4 h, in-clinic, safety and PK/PD assessment.

In Part 2, the subjects were randomized 1:1 to 2 treatment sequences. This part of the study was conducted away from the clinic or hospital. In Sequence 1 (study arm 1) subjects received a single dose of 600 mg of the compound to treat the first eligible HAE attack. Following resolution of this attack, subjects received a single dose of placebo to treat the second eligible HAE attack.

In Sequence 2 (study arm 2) subjects received a single dose of placebo to treat the first eligible HAE attack. Following resolution of this attack, subjects received a single dose of 600 mg of the compound to treat the second eligible HAE attack.

A minimum of 48-hour washout period was required between each dose of study drug.

Laryngeal or facial attacks were not eligible for treatment. HAE attacks must have been treated within the first hour of onset and before reaching severe on the global attack severity scale. Subjects must also have been able to identify the start of a HAE attack. Upon onset of the eligible HAE attack, subjects notified the dedicated study physician or qualified designee with a description of the HAE attack. The dedicated study physician or qualified designee confirmed eligibility of the HAE attack and agreed to study drug being administered. HAE attacks required documentation, on the Subject Diary, of attack location, attack symptoms, time of onset, attack severity, and time of last substantial meal prior to dosing. Subjects took study drug, as instructed, and completed timed assessments of their HAE attack symptoms for a 48 h period as documented below in Table 3. The dedicated study physician or qualified designee contacted the subject within 24 h of the eligible HAE attack to confirm the subject's safety and wellbeing. Subjects were instructed to contact the dedicated study physician or qualified designee in case of any safety concerns. In the case of hypersensitivity, subjects contacted the dedicated study physician or qualified designee or contact the nearest emergency service. The dedicated study physician or qualified designee was available 24 h/day and 7 days/week to receive subject calls.

TABLE 3
Frequency of Subject Assessment

| Time Period | Frequency | Allowed Time |
|---|---|---|
| following Study | of Subject | Window for |
| Drug Administration | Assessment* | Assessment |
| 0 h-4 h | Every 30 min | None |
| 4 h-12 h | Every 1 h | +/−15 min |
| 12 h-24 h | Every 3 h | +/−30 min |
| 36 h | Once | +/−60 min |
| 48 h | Once | +/−60 min |

*In the event that conventional attack treatment was used, the subject performed assessments every 30 min for 4 h following first administration of conventional attack treatment. After this, the subject reverted back to original frequency of assessments based on time of study drug administration.

Subjects returned to the clinic following the first HAE attack, prior to the second HAE attack, to undergo safety checks including adverse event (AE) reporting, vital sign recording, and Subject Diary review.

Once two HAE attacks were treated in Part 2, the subject returned to the clinic to undergo final safety checks including AE reporting, vital sign recording and blood sampling for laboratory safety measurements.

Conventional attack treatment was permitted after 4 h, or earlier as warranted, following study drug intake, provided HAE attack symptoms were judged severe enough by the subject to require treatment as per the subject's usual treatment regimen, or are deemed ineligible for study drug treatment, or were associated with laryngeal or facial symptoms. Prior to use of conventional attack treatment, subjects notified the dedicated study physician or qualified designee who confirmed conventional treatment was appropriate per protocol and subject report of symptom severity. Subjects were permitted to treat their HAE attacks with their conventional attack treatment (pdC1INH or rhC1INH intravenous [iv] or icatibant).

Investigational Medicinal Product:

The compound of formula A—100 mg film-coated tablet. These contained the following excipients: microcrystalline cellulose, croscarmellose sodium, povidone, magnesium stearate; the aesthetic coating contains hypromellose, lactose monohydrate, titanium dioxide and triacetin.

Placebo to the compound 100 mg film-coated tablet. These contained microcrystalline cellulose, colloidal silicon dioxide, sodium starch glycolate, and sodium stearyl fumarate and are film-coated; the aesthetic coating contains hypromellose, lactose monohydrate, titanium dioxide and triacetin.

No study drug dose modifications were allowed in this study.

Number of Subjects:

68 subjects were enrolled into the study to ensure 50 subjects complete the study. 53 subjects completed Part 2.

Population:

The study population included male and female subjects 18 years of age or older with HAE type I or II.

Inclusion Criteria:
1. Male or female adult subjects 18 years of age and older.
2. Confirmed diagnosis of HAE type I or 11 at anytime in the medical history:
   a. Documented clinical history consistent with HAE (subcutaneous or mucosal, nonpruritic swelling episodes without accompanying urticaria) AND
   b. C1-esterase inhibitor (C1-INH) antigen or functional level <40% of the normal level. Subjects with antigen or functional C1-INH level 40-50% of the normal level were enrolled if they also have a C4 level below the normal range and a family history consistent with HAE type I or II.
3. At least 3 documented HAE attacks in the past 93 days, as supported by medical history.
4. Access to and ability to use conventional attack treatment for attacks of HAE.
5. Adequate organ functions as defined below:
   a. Hemoglobin within normal range;
   b. International normalized ratio (INR)<1.2;
   c. Activated partial thromboplastin time (aPTT)≤upper limit of normal (ULN);
   d. Creatinine <1×ULN;
   e. Creatinine clearance (CrCl) ≥60 mL/min;
   f. Alanine aminotransferase (ALT)≤2×ULN;
   g. Aspartate aminotransferase (AST)≤2×ULN;
   h. Total bilirubin≤1.5×ULN;
   i. Leucocytes≤1.5×ULN;
   j. Thrombocytes ≤1.5×ULN.
6. Female of childbearing potential must have agreed to use highly effective birth control from the Screening visit until the end of the trial follow-up procedures.
Highly effective methods of birth control include:
   a. Progestogen-only hormonal contraception associated with inhibition of ovulation: oral/injectable/implantable.
(Hormonal contraception that contains estrogen was excluded per exclusion criterion 3).
   b. Intrauterine device (IUD).
   c. Intrauterine hormone-releasing system (IUS).
   d. Bilateral tubal occlusion.
   e. Vasectomised partner (provided that the partner was the sole sexual partner of the female subject of childbearing potential and that the vasectomised partner received medical assessment of the surgical success).
   f. Sexual abstinence (this method is not acceptable in Switzerland).
   Note: Sexual abstinence was only be considered a highly effective method if it is defined as refraining from heterosexual intercourse. The reliability of sexual abstinence needed to be evaluated in relation to the duration of the clinical trial and the preferred and usual lifestyle of the subject.
7. Females of non-childbearing potential, defined as surgically sterile (status post hysterectomy, bilateral oophorectomy, or bilateral tubal ligation) or post-menopausal for at least 12 months, did not require contraception during the study.
8. Males with female partners of childbearing potential must have agreed to be abstinent or else use a highly effective method of birth control as defined in inclusion criterion 6 from the Screening visit until the end of the trial follow-up procedures.
9. Provide signed informed consent and were willing and capable of complying with study requirements and procedures.

Exclusion Criteria:
1. Any concomitant diagnosis of another form of chronic angioedema, such as acquired C1 inhibitor deficiency, HAE with normal C1-INH (also known as HAE type Ill), idiopathic angioedema, or angioedema associated with urticaria.
2. Current use of C1INH, androgens, lanadelumab or tranexamic acid for HAE prophylaxis.
3. Use of angiotensin-converting enzyme (ACE) inhibitors or any estrogen-containing medications with systemic absorption (such as oral contraceptives or hormonal replacement therapy) within 93 days prior to initial study treatment.
4. Use of androgens (e.g. stanozolol, danazol, oxandrolone, methyltestosterones, testosterone) or antifibrinolytics within 30 days prior to initial study treatment.
5. Use of lanadelumab within 10 weeks prior to initial study treatment.
6. Use of strong CYP3A4/CYP2C9 inhibitors and inducers during participation in the trial.
Note: These medications include but are not limited to the following: cobicistat, conivaptan, itraconazole, ketoconazole, posaconazole, voriconazole, ritonavir, boceprevir, telaprevir, troleandomycin, clarithromycin, carbamazepine, enzalutamide, mitotane, phenytoin, phenobarbital, fluconazole, isoniazid, metronidazole, paroxetine, sulfamethoxazole, rifampicin, St. John's Wort, diltiazem, idelalisib, nefazodone and nelfinavir.
7. Clinically significant abnormal electrocardiogram (ECG) at Visit 1 and pre-dose at Visit 2. This includes, but is not limited to, a QTcF>470 msec (for women) or >450 msec (for men), a PR>220 msec or ventricular and/or atrial premature contractions that are more frequent than occasional and/or occur as couplets or higher in grouping.
8. Any clinically significant history of angina, myocardial infarction, syncope, clinically significant cardiac arrhythmias, left ventricular hypertrophy, cardiomyopathy, or any other cardiovascular abnormality.
9. Any other systemic dysfunction (e.g., gastrointestinal, renal, respiratory, cardiovascular) or significant disease or disorder which, in the opinion of the Investigator, would have jeopardized the safety of the subject by taking part in the trial.
10. History of substance abuse or dependence that would interefere with the completion of the study, as determined by the Investigator.
11. Known lactose allergy or intolerance.
12. Known hypersensitivity to the compound or placebo or to any of the excipients.
13. Participation in an interventional investigational clinical study within 93 days or within 5 half-lives of the last dosing of investigational drug (whichever is longer) prior to initial study treatment.
14. Any pregnant or breast-feeding subject.

Assessments:

Part 1: Blood samples for PK and PD measurements were collected at the following timepoints: Pre-dose (0 h), 15 min, 30 min, 45 min, 1 h, 1.5 h, 2 h, 3 h, and 4 h post-dose. Vital signs (systolic blood pressure [SBP], diastolic blood pressure [DBP], pulse rate [PR], respiratory rate [RR] and body temperature) were measured at pre-dose (0 h), 1 h, and 4 h post-dose. Samples for post-treatment safety laboratory assessments were taken with the 4 h PK/PD samples.

Part 2: Following study drug intake, subject assessments of overall HAE attack severity and change in HAE attack severity took place for a 48 h period as documented in Table 3 above.

Efficacy Variables:

Time to use of conventional attack treatment was assessed. The subject diary captured the efficacy endpoints including time to use of conventional attack treatment and HAE attack severity.

Overall HAE attack severity was assessed on a 5-point Likert scale (5LS) scored as none, mild, moderate, severe and very severe.

Change in HAE attack severity was assessed using a 7-point transition question (7TQ), scored as Much better/Better/A little better/No change/A little worse/Worse/Much worse.

The type of HAE attack symptoms (abdominal pain, skin pain and skin swelling) was each assessed on a 100 mm visual analogue scale (VAS) anchored at 0 (none) and 100 (very severe).

Safety Variables:
AEs, including serious adverse events (SAEs).
Laboratory test results (clinical chemistry, hematology, coagulation, and urinalysis).
Vital signs (SBP, DBP, PR, RR, body temperature).
Physical examination findings.
ECG results.
Pregnancy test (female subjects of child-bearing potential).

Criteria for Evaluation of Efficacy

Primary Efficacy Endpoints:
Time to use of conventional attack treatment.

Secondary Efficacy Endpoints:
Proportion of HAE attacks that progress by one level or more on the 5LS or that require conventional attack treatment within 12 h of study drug.
Time between treatment and (1) progression of global attack severity on the 5LS by one level or more, or (2) use of conventional attack treatment, whichever comes first within 12 h.

Exploratory Endpoints:
Cumulative global attack severity on the 5LS following study drug expressed as area under the curve (AUC) for the compound 600 mg vs. placebo.
Proportion of HAE attacks that require conventional attack treatment.
Proportion of HAE attacks that are rated “worse” or “much worse” on the 7TQ.
Proportion of HAE attacks that are rated “better” or “much better” on the 7TQ.
Time from study drug administration to complete HAE attack resolution (rating of none) on global attack severity scale (5LS).
Time to HAE attack being rated worse or much worse on the 7TQ.
Time to HAE attack being rated better or much better on the 7TQ.

General Statistical Methods and Types of Analyses

Analysis Sets:
Safety set (SAF): Subjects who have taken at least one dose of study drug (including the study drug dose in Part 1).
Full analysis set (for efficacy) (FAS): All randomized subjects who received both doses of study drug in Part 2.
Per protocol set (for efficacy) (PPS): Randomized subjects in Part 2 who received both doses of study drug (the compound) in Part 2 and have no major protocol deviations.
PK/PD analysis set: All subjects for whom PK/PD samples were taken in Part 1.

Sample Size:

A sample size of 50 subjects (25 per sequence) was proposed to provide 90% power for testing at the 5% alpha level (2-sided) for the primary endpoint of time to use of conventional attack treatment. This sample size was derived based upon an assumption that 40% of subjects will use conventional attack treatment while on the control arm while 10% will use conventional attack treatment on the experimental arm and that within subject data has minimal correlation. The assumption of minimal correlation should be a conservative assumption with respect to sample size. 68 subjects were enrolled to ensure that 50 subjects complete the study. 53 subjects completed part 2.

An oversampling by 20% (10 subjects) was proposed to account for subjects that may not complete both treatment periods due to infrequent or ineligible HAE attacks or for subjects who discontinue the trial early, for whatever reason. Thus, study enrolment was considered sufficient to address the primary efficacy hypothesis after 50 subjects have completed both treatment periods. Since further exposure was not required and could be considered unnecessary, ongoing subjects who have not completed both periods were asked to return to the study site and complete Visit 4 (Early Discontinuation visit). Data from all subjects, complete and incomplete, was analyzed in the safety set.

General Considerations:

Individual subject data was presented in subject data listings. Appropriate descriptive statistics was calculated for continuous and categorical data and summarized in tabular format.

Sample Analyses:

AEs were coded using the Medical Dictionary for Regulatory Activities (MedDRA) dictionary (v21.0 or higher) and classified by preferred term and system organ class (SOC). Listings of treatment-emergent adverse events (TEAEs), serious TEAEs, and TEAEs causing premature discontinuation will be provided by sequence group, and further classified by TEAE severity and relationship to study drug.

Efficacy Analyses:

Primary Endpoint

The primary endpoint, time to use of conventional attack treatment, was analyzed using a generalization of Gehan's test proposed by Feingold and Gillespie (1996) (Crossover trials with censored data. Statistics in Medicine 1996; 15(10): 953-967) to reflect the repeat measures on each subject. Subjects were treated as censored if no worsening occurs within 12 h of study drug.

Secondary Endpoints

The proportion of HAE attacks that worsen by one level or more on the 5LS or that require conventional attack treatment within 12 h of study drug was analyzed using Prescott's test (1981) (The comparison of success rates in cross-over trials in the presence of an order effect. Applied Statistics 1981; 30: 9-15) to compare the treatment arms.

A similar approach to that used for the primary endpoint was followed for the analysis of the time between study drug and HAE attack worsening by one level or more on the 5LS or use of conventional attack treatment, whichever comes first within 12 h. In addition to the tests described above, descriptive statistics were presented for the primary, secondary and exploratory endpoints, in each case comparing the compound to placebo, such as:
Cumulative global attack severity on the 5LS following study drug expressed as AUC for the compound 600 mg vs. placebo.
Proportion of HAE attacks that required conventional attack treatment.
Proportion of HAE attacks that were rated “worse” or “much worse” on the 7TQ.
Proportion of HAE attacks that were rated “better” or “much better” on the 7TQ.
Time from study drug administration to complete HAE attack resolution (rating of none) on global attack severity scale (5LS).
Time to HAE attack being rated worse or much worse on the 7TQ.
Time to HAE attack being rated better or much better on the 7TQ.

PK Analysis:

Non-compartmental PK parameters included maximum concentration in plasma (Cmax), time to reach Cmax in plasma (tmax), and area under the curve from time 0 to last sample (AUC0-t). Compartmental PK modelling described the PK of the compound and generate underlying Cmax, tmax, AUC, apparent clearance (CL/F), apparent volume of distribution (Vd/F) and estimated terminal elimination half-life (t½).

The PK parameters of the compound were determined from the individual concentration versus time data using Phoenix WinNonlin. In case of a deviation from the theoretical time, the actual time of blood sample was used in the calculation of the derived PK parameters. Individual concentrations and derived PK parameters of the compound in plasma were listed and summarized for each treatment. Individual and geometric mean concentration-time data were plotted on linear and semi-logarithmic scales.

PD Analysis:

The compound's effect on plasma kallikrein (PKa) activity was analyzed using two exploratory measures of PKa enzyme activity in plasma:
An assay to determine inhibition of exogenously activated plasma kallikrein enzyme activity from plasma samples obtained before and after receiving the compound.
An assay to measure the level of protection of cleavage of high molecular weight kininogen (HK) substrate (contained in whole plasma) from plasma kallikrein enzyme activity.

The PD were summarized for each treatment. Individual and mean data were provided as a report addendum located in the appendix of the final Clinical Study Report.

Preliminary PK Data from Part 1 of the Study:

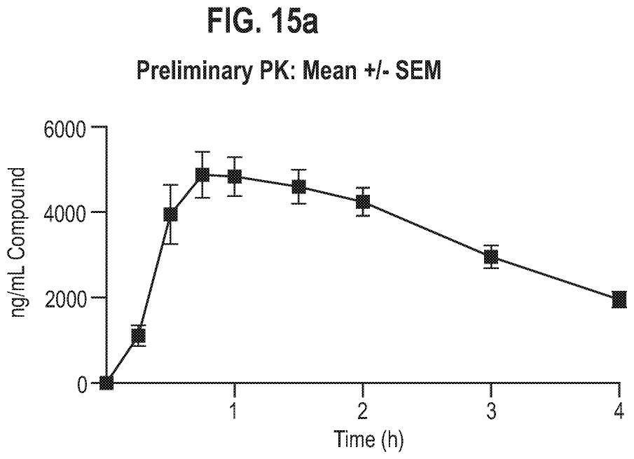

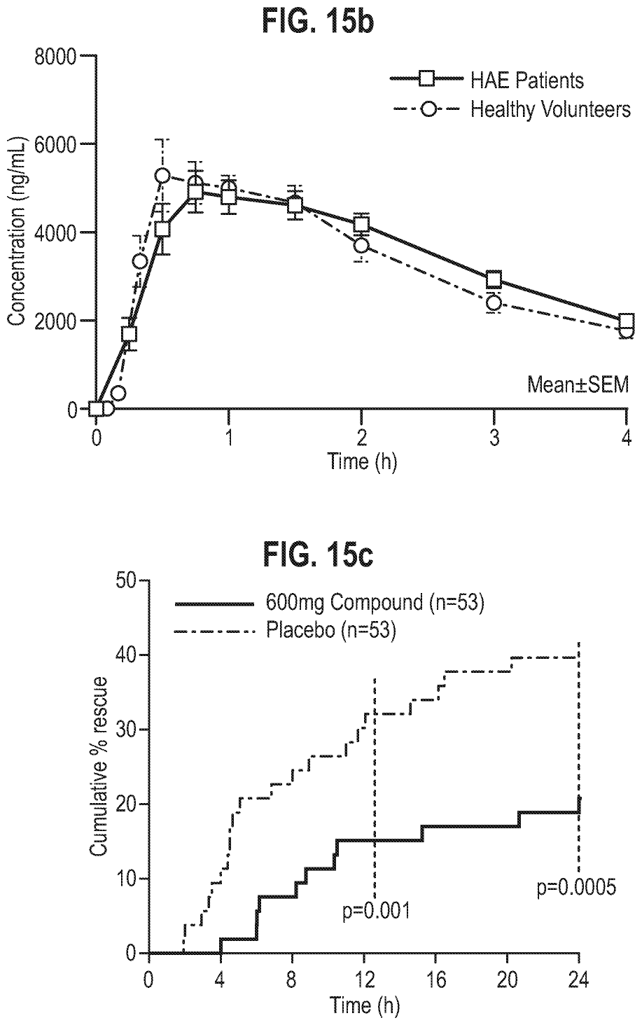

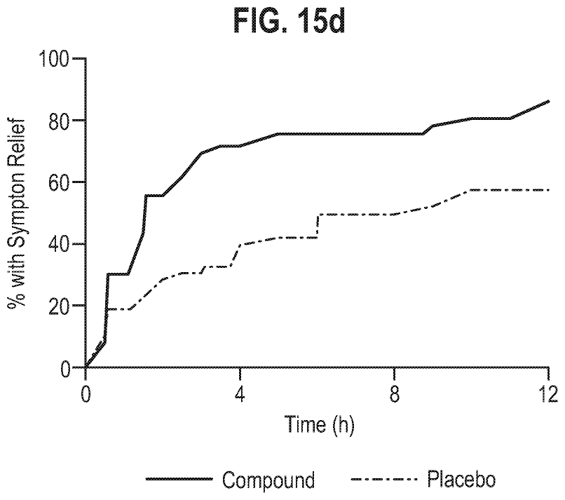

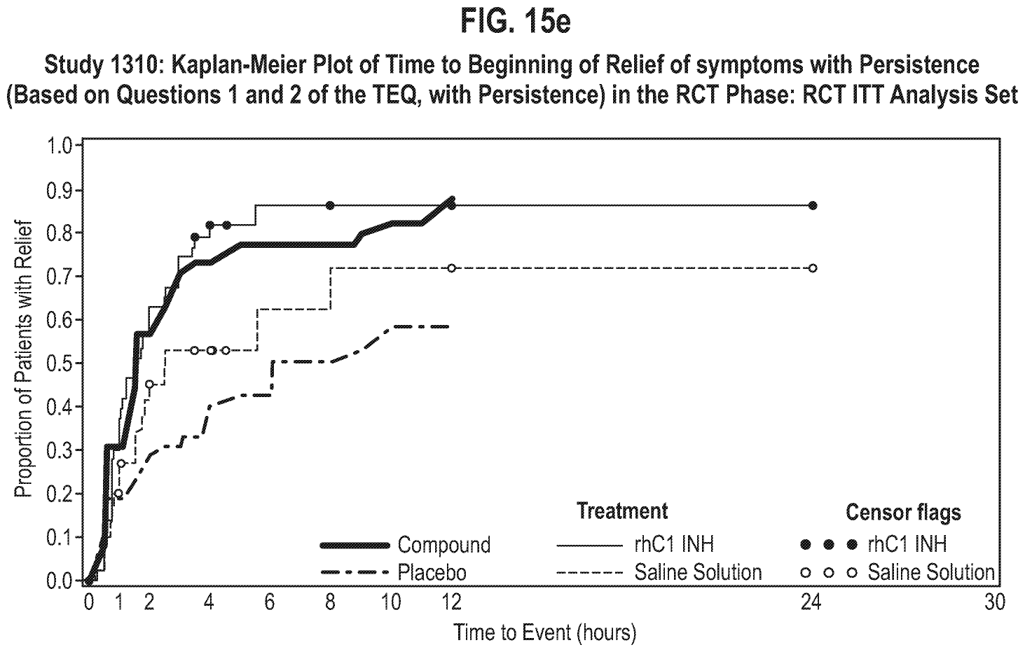

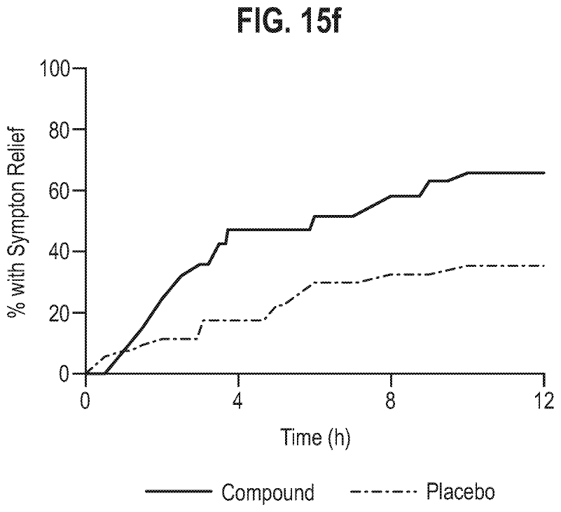

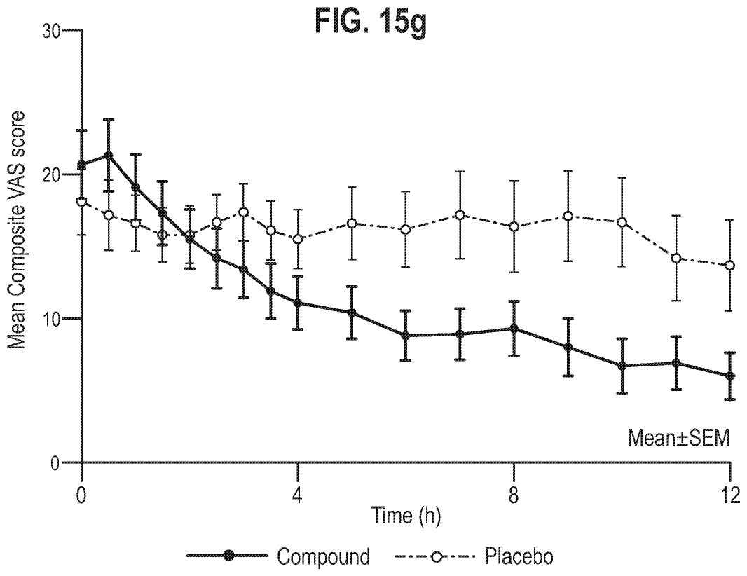

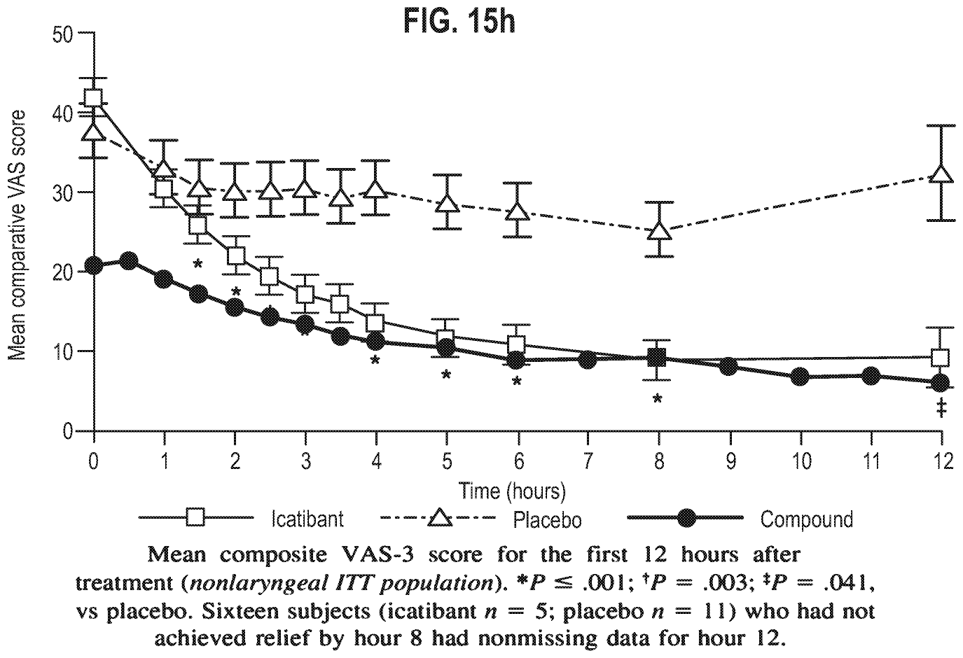

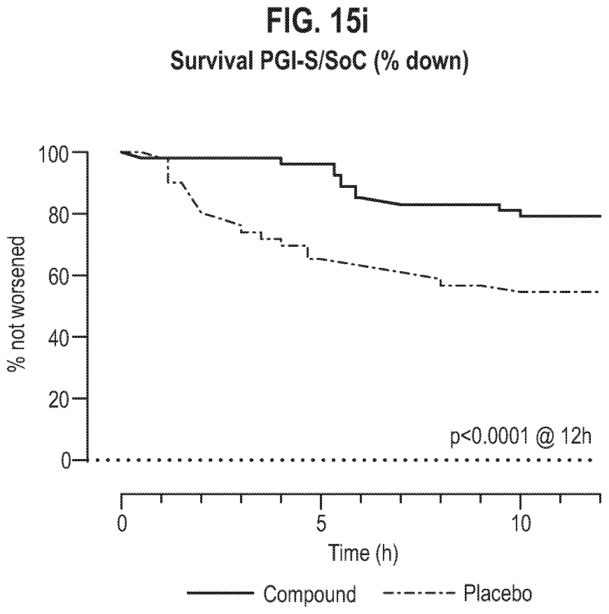

Preliminary PK data from 27 HAE patients was collated and analysed, and are shown in Table 4 and FIG. 15a.

TABLE 4

| | Cmax (ng/ml) |
|---|---|
| | Mean (95% Cl) |
| Dose | n = 27 |
| 600 mg | 5907 (4913, 6901) |

Thus, these preliminary results show that the compound of Formula A demonstrates a pharmacokinetic profile that is suitable for on-demand oral administration in HAE patients.

FIG. 15b shows the complete PK profile of HAE patients from part 1, overlaid with PK data from healthy volunteers.

Efficacy Results from Part 2 of the Study

Primary Endpoint

Attacks treated with the compound significantly reduced use of rescue (p=0.0010) with 15.1% of compound treated attacks rescued compared to 30.2% on placebo at 12 hours. This efficacy benefit of the compound was maintained at 24 hours (p=0.0005). At 24 hours, use of rescue medication (p=0.0005) was 20.8% for the compound versus 39.6% on placebo.

FIG. 15c shows the cumulative % of patients that used rescue medication within 12 hours of being administered the compound.

Secondary Endpoints

The compound significantly reduced time to onset of symptom relief (p=<0.0001) on a Patient Global Impression of Change scale (PGI-C) (also known as the 7TQ) with a median time of 1.6 hours versus 9 hours for attacks treated with placebo. This demonstrates that treatment with the compound achieved symptom relief more quickly than placebo.

Within 12 hours of study drug, 83.0% of attacks treated with the compound were rated “a little better” or higher (when assessed according to the 7TQ), compared to 50.9% of attacks treated with Placebo (p<0.005). Within 24 hours of study drug, 84.9% of attacks treated with the compound were rated “a little better” or higher (when assessed according to the 7TQ), compared to 64.2% of attacks treated with Placebo (p<0.05).

Within 12 hours of study drug, 58.7% of attacks treated with the compound were rated “better” or higher (when assessed according to the 7TQ), compared to 35.8% of attacks treated with placebo (p=0.0319). Within 24 hours of study drug, 67.9% of attacks treated with the compound were rated “better” or higher (when assessed according to the 7TQ), compared to 47.2% of attacks treated with placebo (p=0.0593).

Treatment with the compound led to a statistically significant shortening of the median time for attacks to be rated “better” or higher (when assessed according to the 7TQ), with a median time of 5 hours for treatment with the compound (p=0.0003) compared to a median time of 15 hours for placebo (p=0.0036).

Of the attacks that achieved a rating of “better” or higher within 24 hours (when assessed according to the 7TQ), 6.1% used rescue medication, 66.7% achieved attack resolution when assessed according to the PGI-S, and 77.6% achieved attack resolution when assessed by VAS. On the contrary, of the attacks that did not achieve a rating of “better” or higher within 24 hours (when assessed according to the 7TQ), 63.8% used rescue medication, 4.3% achieved attack resolution when assessed according to the PGI-S, and 5.1% achieved attack resolution when assessed by VAS.

FIG. 15d shows the cumulative % of patients with symptom relief (as assessed using the 7TQ method) within 12 hours of being administered the compound. FIG. 15e compares time to symptom relief data for the compound with known clinical data for approved injectable product Ruconest® (see Charles M. Maplethorpe, MD, PhD. Clinical Reviewer. Summary Basis of Approval, Recombinant C1 Esterase Inhibitor, STN: 125495/0).

Attacks treated with the compound achieved symptom relief more quickly than placebo treated attacks (p<0.0001) when assessed using a composite Visual Analogue Scale (VAS) score (abdominal pain, skin pain, and skin swelling) with a median time to symptom relief of 6 hours versus >12 hours for placebo. Again, this demonstrated that treatment with the compound achieved symptom relief more quickly than placebo.

FIG. 15f shows the cumulative % of patients with symptom relief (as assessed using the composite VAS score) within 12 hours of being administered the compound.

Attacks treated with the compound reduced the severity of the HAE attack (as assessed using the composite VAS score) over 12 hours (p=0.0008) and 24 hours (p=0.0005). Within 12 hours of study drug, 79.2% of attacks treated with the compound do not worsen, compared to 54.7% of attacks treated with Placebo.

FIG. 15g shows the mean composite VAS score within 12 hours of being administered the compound. FIG. 15g is time- and baseline-adjusted AUC and excludes assessments post-rescue medication. FIG. 15h compares the mean composite VAS score for patients treated with the compound with known clinical data for approved injectable product Firazyr® (active ingredient, icatibant) (see Lumry et al., Ann Allergy Asthma Immunol. 2011; 107:529-537).

Within 12 hours of oral administration, the compound significantly increased the number of attacks stabilized or improved when assessed by a Patient Global Impression of Severity scale (PGI-S) (also known as the 5LS) or use of rescue (p<0.0001).

FIG. 15i shows the % of HAE attacks that did not worsen (as assessed using the 5LS method) within 12 hours of being administered the compound. As noted above, within 12 hours of study drug, 79.2% of attacks treated with the compound do not worsen, compared to 54.7% of attacks treated with Placebo. Within 24 hours of study drug, 69.8% of attacks treated with the compound do not worsen, compared to 50.9% of attacks treated with Placebo.

Within 12 hours of study drug, 37.7% of attacks treated with the compound improved to “none” when assessed using the PGI-S (also called the 5LS method), compared to 18.9% of attacks treated with Placebo. Within 24 hours of study drug, 52.8% of attacks treated with the compound improved to “none”, compared to 26.4% of attacks treated with Placebo.

Results from other measured endpoints were as follows:

| | | | |
|---|---|---|---|
| Change in Attack Status | | | |
| (12 hours and 24 hours | Compound | Placebo | P |
| after administration) | N = 53 | N = 53 | Value |

PGI-C (7TQ)

| | | | |
|---|---|---|---|
| Median time to HAE attack | | | |
| being (1) rated a little | | | |
| worse or higher for two | | | |
| consecutive time points, | | | |
| or (2) use of conventional | | | |
| attack treatment, | | | |
| whichever comes first, | | | |
| within 12 hours and 24 | | | |
| hours of study drug | | | |
| administration, hours | | | |
| At 12 hours | >12 | 4.7 | <0.0001 |
| At 24 hours | >24 | 4.7 | 0.0003 |
| Median time to HAE | | | |
| attack being rated a little | | | |
| worse or higher for two | | | |
| consecutive time points, | | | |
| within 12 hours and | | | |
| 24 hours of study drug | | | |
| administration, hours | | | |
| At 12 hours | >12 | 9.0 | 0.0004 |
| At 24 hours | >24 | 9.0 | 0.0103 |
| Proportion of HAE attacks | | | |
| that were stable or | | | |
| improved within 12 hours | | | |
| and 24 hours of study | | | |
| drug administration, n (%) | | | |
| At 12 hours | 48 (90.6) | 35 (66.0) | 0.0021 |
| At 24 hours | 45 (84.9) | 34 (64.2) | 0.0189 |

PGI-S (5LS)

| | | | |
|---|---|---|---|
| Proportion of HAE attacks | | | |
| that improved by 1 level | | | |
| or more from baseline | | | |
| within 12 hours and 24 hours of | | | |
| study drug administration, n (%) | | | |
| At 12 hours | 30 (56.6) | 16 (30.2) | 0.0109 |
| At 24 hours | 35 (66.0) | 20 (37.7) | 0.0069 |
| Median time to improvement | | | |
| by one level or more | | | |
| from baseline within 12 | | | |
| hours and 24 hours of | | | |
| study drug administration, hours | | | |
| At 12 hours | 9.0 | >12 | 0.0002 |
| At 24 hours | 9.0 | >24 | 0.0008 |
| Proportion of patients with | | | |
| HAE attack resolution | | | |
| (rating of none) within 12 | | | |
| hours and 24 hours of | | | |
| study drug administration, n (%) | | | |
| At 12 hours | 20 (37.7) | 10 (18.9) | 0.0622 |
| At 24 hours | 28 (52.8) | 14 (26.4) | 0.0123 |
| Median time to HAE attack | | | |
| resolution (rating of | | | |
| none) within 12 hours and | | | |
| 24 hours of study drug | | | |
| administration, hours | | | |
| At 12 hours | >12 | >12 | 0.0064 |
| At 24 hours | 18.0 | >24 | 0.0021 |

VAS

| | | | |
|---|---|---|---|
| Proportion of HAE attacks | | | |
| with symptom relief | | | |
| (50% reduction from baseline | | | |
| in composite VAS for | | | |
| three consecutive time points) | | | |
| within 12 hours and | | | |
| 24 hours of study drug, n (%) | | | |
| At 12 hours | 33 (62.3) | 16 (30.2) | 0.0014 |
| At 24 hours | 39 (73.6) | 20 (37.7) | 0.0006 |

Of the attacks treated with the compound of Formula A, 31.0% and 69.0% were categorized as abdominal and peripheral attacks, respectively, and 77.8% of abdominal attacks and 77.5% of peripheral attacks achieved symptom relief (PGI-C score of at least “A little better” within 12 hours). Median time to symptom relief was 1.5 hours for abdominal attacks and 2.5 hours for peripheral attacks). Baseline attack severity was 1.7 for abdominal and 1.5 for peripheral, where “baseline attack severity” is assessed by assigning numeric values from 0 to 4 to align with categorical PGI Severity scale (PGI-S) scores (from “None” to “Very Severe”). A total of 61.1% of abdominal attacks and 50.0% of peripheral attacks achieved attack resolution within 24 hours.

Safety

There were no serious adverse events reported in the trial and no patients withdrew due to adverse events. In the open-label phase (Part 1), 8 on-treatment drug-related treatment emergent adverse events (TEAE) were experienced by 5 patients. In the crossover phase of the trial (Part 2), 3 on-treatment drug-related TEAEs were experienced by 3 patients (5.2%) following administration of the compound, and 2 on-treatment drug-related TEAEs were experienced by 2 patients (3.6%) following administration of placebo. These data are shown below.

| | Part 1 | Part 2 | Part 2 |
|---|---|---|---|
| | KVD900 | KVD900 | Placebo |
| | N = 68 | N = 58 | N = 55 |
| Total (events/patients) | 8/5 (7.4%) | 3/3 (5.2%) | 2/2 (3.6%) |
| Gastrointestinal Disorders | 1/1 (1.5%) | 1/1 (1.7%) | 1/1 (1.8%) |
| Abdominal Pain Upper | — | 1/1 (1.7%) | — |
| Anal Incontinence | — | — | 1/1 (1.8%) |
| Nausea | 1/1 (1.5%) | — | — |
| General Disorders | 1/1 (1.5%) | — | — |
| Malaise | 1/1 (1.5%) | — | — |
| Musculoskeletal Disorders | 1/1 (1.5%) | 1/1 (1.7%) | — |
| Back Pain | 1/1 (1.5%) | 1/1 (1.7%) | — |
| Nervous System Disorders | 3/3 (4.4%) | 1/1 (1.7%) | 1/1 (1.8%) |
| Dizziness | 1/1 (1.5%) | — | — |
| Headache | 2/2 (2.9%) | 1/1 (1.7%) | 1/1 (1.8%) |
| Vascular Disorders | 2/2 (2.9%) | — | — |
| Flushing | 2/2 (2.9%) | — | — |

### Example 7—Phase 3 Study of the Compound of Formula A

A Randomized, Double-Blind, Placebo-Controlled, Phase 3, Three-way Crossover Trial to Evaluate the Efficacy and Safety of Two Dose Levels of the compound of Formula A (“the compound” or “study drug”), an Oral Plasma Kallikrein Inhibitor, for On-Demand Treatment of Angioedema Attacks in Adolescent and Adult Patients with Hereditary Angioedema Type I or II.

Objectives:

Primary Objective:
To demonstrate the clinical efficacy of the compound compared with placebo for the on-demand treatment of HAE attacks.

Secondary Objective:
To investigate the safety and tolerability of the compound.

Setup:

This will be a double-blind, randomized, placebo-controlled, multicentre clinical trial in patients 12 years or older with HAE type I or II. Patients will be randomized to 6 treatment sequences in a 3-way crossover design. Eligible attacks will initially be treated with a single dose of placebo, 300 mg, or 600 mg of the compound per attack with a minimum 48-hour washout period between each eligible attack and last dose of compound or conventional on-demand treatment. If needed (as determined by the patient), a second dose of the study drug may be administered for each attack.

The estimated duration of this trial for each randomized patient will be approximately 25 weeks from screening through the final visit and includes the treatment of 3 eligible attacks during the treatment period.

This trial will be conducted at HAE treatment centres on an outpatient basis and will comprise in-clinic and televisits. A televisit can be conducted via a telephone call or a via an interactive audio/video system. If an in-clinic visit cannot be conducted (e.g. in the event of a pandemic or other reason that prevents the patient from attending the in-clinic visit) home health visits will be used to perform these visits if permitted by the relevant regulatory authority, site's Ethics Committee (EC)/Institutional Review Board (IRB), local regulations, and the patient via informed consent. The home visit will be performed by an appropriately delegated home healthcare service provider. Information captured during a home health visit will mirror that captured in an in-clinic visit.

Screening Visit

Eligible patients ≥12 years old will undergo a screening assessment for trial inclusion. All patients will provide informed consent or assent prior to any trial-related procedures being performed. Informed consent and assent may be collected through e-consent if allowed through country and site regulations.

During the visit, a physical exam, 12-lead electrocardiogram (ECG), laboratory tests (including diagnostic testing for HAE), and other assessments will be performed.

Site personnel will train patients on the information they will be expected to provide in the electronic diary (eDiary) and the use of the study drug.

Randomization Visit

Within 4 weeks of the Screening Visit, patients will participate in a Randomization Visit. Patients will be assigned to receive 3 treatments in randomized, double-dummy blinded, crossover fashion based on their assignment to 1 of 6 treatment sequences. Randomization will be stratified by whether the patient enters the trial taking only conventional on-demand treatment vs. on a stable dose and regimen of long-term prophylactic treatment. Randomization will occur in a 1:1:1:1:1:1 ratio using a permuted-block randomization method to ensure a balanced assignment to each treatment sequence. Each patient will receive the following treatments:
300 mg of the compound (1×300 mg tablet plus 1 matching placebo tablet)
600 mg of the compound (2×300 mg tablets)
2 matching placebo tablets

Patients will treat each eligible attack with up to 2 doses of the study drug, administered at least 3 hours apart. The second dose, if taken, will be the same assigned treatment as the first dose.

In this trial, the study drug will be shipped directly to the patients via a courier service or will be dispensed at the trial clinic as required by local regulations or per the site's local practice, as described in the Pharmacy Manual.

Treatment of Eligible HAE Attacks

Patients will treat 3 separate, eligible HAE attacks with their assigned study drug treatment for that attack. For an HAE attack to be considered eligible for treatment with the study drug, the attack must meet the following criteria:
The attack is not a severe laryngeal attack.
Patient must be able to identify the start time of the attack.
At least 48 hours have elapsed since patient has used conventional on-demand treatment or the study drug to treat an HAE attack.
Patient must be able to complete at least the first 4 hours of eDiary assessments following the first administration of the study drug.
Post-attack televisit has been completed for the previous eligible attack (applicable to eligible attacks 2 and 3 only).

Eligible attacks should initially be treated with a single administration of the study drug. Patients will be encouraged to treat as soon as possible after the start of the attack.

If needed (as determined by the patient), a second dose of the study drug may be administered for each attack, as follows:

Non-Laryngeal Attacks

For each eligible HAE attack, a second dose of the study drug may be taken:
after 3 hours if HAE attack symptoms are considered severe enough by the patient to require a second dose of the study drug.

After the second dose of the study drug, conventional on-demand treatment may be taken:
after 1 hour if HAE attack symptoms are considered severe enough by the patient to require treatment with conventional treatment.

If symptoms progress to airway involvement, patients may treat with conventional on-demand treatment at any time.

Laryngeal Attacks

After the first dose of the study drug, conventional on-demand treatment may be taken at any time:
if HAE attack symptoms worsen or if HAE attack symptoms are considered severe enough by the patient to require immediate treatment.

Attacks that do not meet eligibility may be treated with conventional on-demand treatment per the patient's usual treatment regimen.

Conventional on-demand treatments may include plasma derived C1-inhibitor (pdC1-INH) intravenous (iv), recombinant human C1-esterase inhibitor (rC1-INH) iv, icatibant sc, or ecallantide sc.

Patients with safety or tolerability concerns will contact the Investigator or designee as soon as possible or the nearest emergency service as appropriate.

Study Call Center

After the first dose of the study drug and prior to the second dose or conventional on-demand treatment, patients will be required to call a designated Study Call Center to be reminded of the repeat dosing criteria. Patients are to contact the Call Center:
1. After the first dose of the study drug
2. Prior to a second dose of the study drug
3. Prior to a dose of conventional on-demand treatment.

The Call Center staff will remind patients of the rules for re-dosing and the eDiary assessment requirements. The Call Center will not collect any data during the call.

Patient eDiary

For each HAE attack treated with the study drug, patients will record information in an eDiary, including attack location, attack symptoms, date/time of onset, attack severity, time of second study drug dose, if applicable, and use of conventional on-demand treatment, if applicable. Patients will complete timed assessments of their HAE attack through 48 hours as documented in Table 5. Patients should complete all timed diary assessments except during sleep; however, patients must complete at least the first 4 hours of diary assessments following the first administration of the study drug.

TABLE 5
Frequency of Patient Assessment

| Time Period | Frequency | Allowed Time | |
|---|---|---|---|
| following Study | of Patient | Window for | |
| Drug Administration | Assessment* | Assessment | |
| 0 to 4 hours | Every 0.5 hours | ±0.25 | hour |
| 5 to 12 hours | Every 1 hour | ±0.5 | hour |
| 14 to 24 hours | Every 2 hours | ±1 | hour |
| 25 to 48 hours | Every 12 hours | ±3 | hours |

*If a second dose of IMP or additional doses of conventional on-demand treatment are needed, patients will complete diary assessments prior to taking each additional dose. After re-dosing, the planned post-dose diary assessments will then continue through 48 hours after first dose of the study drug.

Post-Attack Televisit

A televisit (between the site staff and the patient) will be completed following each administration of the study drug to ensure the safety and wellbeing of the patient, to confirm the study drug accountability, to review the patient diary (and retrain, if necessary), and to undergo an adverse event (AE) and concomitant medication review. The televisit will occur by the next working day after the completion of the patient diary (visit window: +1 week).

Final Visit/Early Termination

Once 3 HAE attacks have been treated, or upon early termination, the patient will return to the clinic as soon as possible within 1 week for an in-clinic visit to undergo final safety checks including AE reporting, vital sign recording, and blood sampling for laboratory safety measurements. Whenever possible, this visit should be completed prior to starting any new medication or treatment.

Investigational Medicinal Product:

The compound of Formula A—300 mg film-coated tablet. These contained the following excipients: microcrystalline cellulose, croscarmellose sodium, povidone, magnesium stearate; the aesthetic coating contains polyvinyl alcohol, titanium dioxide and polyethylene glycol 3350.

Placebo for the compound tablet. These contained microcrystalline cellulose, croscarmellose sodium, povidone, magnesium stearate and are film-coated; the aesthetic coating contains polyvinyl alcohol, titanium dioxide and polyethylene glycol 3350.

No study drug dose modifications will be allowed in this study.

Tablets must be swallowed whole and are not to be crushed or modified in any way. Eligible attacks should initially be treated with a single administration of the study drug as soon as possible after the start of the attack. If needed (as determined by the patient), a second dose of the study drug may be administered for each attack.

Number of Patients:

Approximately 114 patients will be enrolled into the trial to ensure approximately 84 patients (including a minimum of 12 adolescents) complete the trial.

The trial population will comprise 2 subsets: (1) patients who enter the trial taking only conventional on-demand treatment and (2) patients who enter the trial on a stable dose and regimen of long-term prophylactic treatment

Population:

The trial population will include male and female patients 12 years of age and older with a confirmed diagnosis of HAE type I or II.

The trial population will include a subset of patients who enter the trial taking only conventional on demand treatment and a subset of patients who enter the trial on a stable dose and regimen of long-term prophylactic treatment.

Inclusion Criteria:
1. Male or female patients 12 years of age and older.
2. Confirmed diagnosis of HAE type I or II at any time in the medical history:
   a. Documented clinical history consistent with HAE (sc or mucosal, nonpruritic swelling episodes without accompanying urticaria) and either:
      i. Diagnostic testing results obtained during the Screening Period that confirm HAE type I or II: C1-INH functional level <40% of the normal level. Patients with functional C1-INH level 40-50% of the normal level may be enrolled if they also have a C4 level below the normal range. Patients may be retested during the Screening Period if results are incongruent with clinical history or believed by the Investigator to be confounded by recent prophylactic or therapeutic C1-INH use, or
      ii. Documented genetic results that confirm known mutations for HAE type I or II.
3. Patient has access to and ability to use conventional on-demand treatment for HAE attacks.
4. If a patient is receiving long-term prophylactic treatment with one of these medicines indicated for HAE: intravenous (iv) or sc plasma-derived C1-INH, and/or lanadelumab, they must be on a stable dose and regimen for at least 3 months prior to the Screening Visit and be willing to remain on a stable dose and regimen for the duration of the trial.
5. Patient's last dose of attenuated androgens was at least 28 days prior to randomization.
6. Patient has had at least 2 documented HAE attacks within 3 months prior to randomization.
7. Patients must meet one of the following contraception requirements as follows:
   a. Female patients who are fertile and heterosexually active must agree to use contraception from the Screening Visit until the Final or Early Termination (ET) Visit. Acceptable methods of contraception include one or more of the following:
      i. Progestogen-only hormonal contraception associated with inhibition of ovulation: oral/injectable/implantable (hormonal contraception that contains estrogen including ethinylestradiol is excluded per Exclusion 4).
      ii. Intrauterine device.
      iii. Intrauterine hormone-releasing system.
      iv. Bilateral tubal occlusion.
      v. Vasectomized partner (provided that the partner is the sole heterosexual partner of the female patient of childbearing potential and that the vasectomized partner has received medical assessment of surgical success).
      vi. Male or female condom.
      vii. Cap, diaphragm, or sponge with spermicide.
   b. Patients who are not fertile or not heterosexually active, as defined below, do not require contraception. If the patient's status changes during the course of the trial, they will be required to meet the requirements specified in Inclusion Criterion 7a.
      i. Female patients who refrain from heterosexual intercourse during the trial if the reliability of the heterosexual abstinence has been evaluated in relation to the duration of the clinical trial and is the preferred and usual lifestyle of the patient.
      ii. Female patients who are surgically sterile (e.g. status post hysterectomy, bilateral oophorectomy, or bilateral tubal ligation) or post-menopausal for at least 12 months.
      iii. Female patients who are premenarche and remain premenarcheal until the end of the trial.
      iv. Male patients (including female partners).
8. Patients must be able to swallow trial tablets whole.
9. Patients, as assessed by the Investigator, must be able to appropriately receive and store the study drug, and be able to read, understand, and complete the electronic diary (eDiary).
10. Investigator believes that the patient is willing and able to adhere to all protocol requirements.
11. Patient provides signed informed consent or assent (when applicable). A parent or legally authorized representative must also provide signed informed consent when required.

### Exclusion Criteria:

1. Any concomitant diagnosis of another form of chronic angioedema, such as acquired C1-inhibitor deficiency, HAE with normal C1-INH (previously known as HAE type Ill), idiopathic angioedema, or angioedema associated with urticaria.
2. A clinically significant history of poor response to bradykinin receptor 2 (BR2) blocker, C1-INH therapy or plasma kallikrein inhibitor therapy for the management of HAE, in the opinion of the Investigator.
3. Use of angiotensin-converting enzyme (ACE) inhibitors after the Screening Visit or within 7 days prior to randomization.
4. Any estrogen containing medications with systemic absorption (such as oral contraceptives including ethinylestradiol or hormonal replacement therapy) within 7 days prior to the Screening Visit.
5. Use of strong cytochrome P450 3A4 CYP3A4 inhibitors and inducers during participation in the trial, starting within 5 half-lives of the Screening Visit.
Note: These medications include but are not limited to the following:
Inhibitors: boceprevir, clarithromycin, cobicistat, dasabuvir, denoprevir, elvitegravir, idelalisib, indinavir, itraconazole, ketoconazole, lopinavir, nefazodone, nelfinavir ombitasvir, paritaprevir, posaconazole, ritonavir, saquinavir, telaprevir, telithromycin, tipranavir, troleandomycin, and voriconazole.
Inducers: apalutamide, carbamazepine, enzalutamide, mitotane, phenytoin, rifampin, St. John's Wort.
6. Inadequate organ function, including but not limited to:
   a. Alanine aminotransferase (ALT) >2× upper limit of normal (ULN)
   b. Aspartate aminotransferase (AST) >2×ULN
   c. Bilirubin direct >1.25×ULN
   d. International normalized ratio (INR) >1.2
   e. Clinically significant hepatic impairment defined as a Child-Pugh B or C.
7. Any clinically significant comorbidity or systemic dysfunction, which in the opinion of the Investigator, would jeopardize the safety of the patient by participating in the trial.
8. History of substance abuse or dependence that would interfere with the completion of the trial, as determined by the Investigator.
9. Known hypersensitivity to the compound or placebo or to any of the excipients.
10. Prior participation in the phase 2 trial.
11. Participation in any gene therapy treatment or trial for HAE.
12. Participation in any interventional investigational clinical trial, including an investigational COVID 19 vaccine trial, within 4 weeks of the last dosing of investigational drug prior to screening.
13. Any pregnant or breastfeeding patient.

Assessments:

Efficacy Variables:
Patient global impression of change (PGI-C) scored on a 7-point rating scale as much better, better, a little better, no change, a little worse, worse, much worse.
Patient global impression of severity (PGI-S) scored on a 5-point rating scale as none, mild, moderate, severe, and very severe.
Visual analog scale (VAS) anchored at 0 (none) and 100 (very severe) for abdominal pain, skin pain, and skin swelling.
Modified General Anxiety—Numeric Rating Scale (GA-NRS) scored on an 11-point scale anchored at 0 (not at all anxious) and 10 (extremely anxious).
Use of conventional on-demand treatment.

Safety Variables:
Adverse events, including serious adverse events.
Laboratory test results.
12-lead ECG.
Vital signs.
Physical examination findings.

Criteria for Evaluation of Efficacy

Primary Efficacy Endpoints:
PGI-C: Time to beginning of symptom relief defined as at least “a little better” (2 time points in a row) within 12 hours of the first study drug administration.

Key Secondary Efficacy Endpoints:
PGI-S: Time to first incidence of decrease from baseline within 12 hours of the first study drug administration.
PGI-S: Time to HAE attack resolution defined as “none” within 24 hours of the first study drug administration.

Secondary Efficacy Endpoints:
PGI-C: Proportion of attacks with beginning of symptom relief defined as at least “a little better” (2 time points in a row) within 4 hours and within 12 hours of the first study drug administration.
PGI-C: Time to at least “better” within 12 hours of the first study drug administration.
PGI-S: Time to first incidence of decrease from baseline within 24 hours of the first study drug administration.
Composite VAS: Time to at least a 50% decrease from baseline (3 time points in a row) within 12 hours and within 24 hours of the first study drug administration.

Exploratory Endpoints:
GA-NRS: Cumulative GA-NRS expressed as area under the curve over 12 and 24 hours of the first study drug administration.

General Statistical Methods and Tyes of Analyses

Analysis Sets:
Safety Set will include all patients who receive at least one dose of trial medication.
Full Analysis Set (FAS) will include all randomized patients who receive trial medication from at least two periods (including placebo period) after respective qualifying HAE attack. If one or more patient(s) received incorrect trial medication, data summarized using the FAS will be presented according to the randomized treatment. The FAS population will be the population for confirmatory efficacy analyses.
Per-protocol Set (PPS) includes all patients from FAS who receive all three trial medications, complete scheduled assessments, and who do not have pre-defined protocol deviations that may affect primary efficacy endpoint.
Subgroup analysis sets
On-demand Full Analysis Set on-demand FAS) will include FAS patients who enter the trial taking only conventional on-demand treatment.
Prophylaxis Full Analysis Set (prophylaxis-FAS) will include FAS patients who enter the trial on a stable dose and regimen of long-term prophylactic treatment.

Sample Size:
Approximately 114 patients will be randomized into the trial to ensure approximately 84 patients (including a minimum of 12 adolescents) complete the trial. The trial population will comprise 2 subsets: (1) patients who enter the trial taking only conventional on-demand treatment and (2) patients who enter the trial on a stable dose and regimen of long-term prophylactic treatment
   1. A sample size of 66 patients completing the trial would provide 90% power for testing each pairwise comparison (the compound versus placebo) at the 2.5% alpha level (2-sided) for the primary endpoint of time to beginning of symptom relief of the HAE attack as defined by PGI-C as at least “a little better” for 2 time points in a row within 12 hours of the first study drug administration. This sample size is derived based upon an assumption that median time to symptom relief of the HAE attack is 1.6 hours in active dose arm and 9 hours in placebo arm from the Phase 2 trial. It is assumed that patients will begin the trial together and will be followed for the same period of time, 49% of patients in control group and 17% of patients in the compound dose group are assumed to be lost-to-follow-up (right-censored). Taking a conservative approach and using simulation-based procedure for power calculations for a parallel group design, the two-sided two-group survival comparison Gehan Wilcoxon test has approximately 90% power to detect a median time ratio of 5.6 (9/1.6) with a target 2-sided significance level of 2.5% when there are 84 patients in each treatment group.
   2. The treatment effect observed in the Phase 2 trial (Example 6) is assumed to be representative of the entire population in this study. However, additional trial populations will be enrolled (eg, adolescent patients and patients that enter the trial on a stable dose and regimen of long-term prophylactic treatment where the treatment effect has not been previously characterized. Therefore a total of 84 patients completing the trial is proposed. This conservative approach increases the likelihood of maintaining at least 90% power in the event that the true treatment effect in this study population is different to that observed in the phase 2 trial.

Assuming there is approximately 30% dropout or non-completion rate, consistent with the Phase 2 trial, the oversampling by 30 patients (84+30=114) is proposed to account for patients that may not complete all treatment periods due to infrequent or ineligible HAE attacks or for patients who discontinue the trial early, for whatever reason.

General Considerations:

Continuous data will be summarized by treatment group using descriptive statistics (number, arithmetic mean, median, standard deviation, minimum, and maximum). Categorical data will be summarized by treatment group using frequency tables (frequencies and percentages).

All analyses will be carried out using SAS Version 9.4 or higher or using other validated software.

Sample Analyses:

The primary efficacy endpoint confirmatory analysis will have Bonferroni multiplicity adjustment for multiple dose levels, therefore pairwise comparison tests will be 2-sided with an alpha of 0.025. The analysis of the secondary or exploratory endpoints will not have multiplicity adjustments.

There are 3 treatment groups in the trial:
1. 300 mg of the compound
2. 600 mg of the compound
3. Placebo

Two pairwise comparisons will be performed: 300 mg of the compound versus placebo and 600 mg of the compound versus placebo.

Confirmatory efficacy analysis will be performed on the Full Analysis Set.

Efficacy Analyses:

A fixed sequence closed testing procedure will be followed. In a fixed sequence closed testing procedure the formal inferential testing can proceed to the next step only when statistical significance is declared in the current step. If the testing sequence is stopped, the remaining endpoints in the testing sequence will be considered exploratory. The fixed testing procedure will be employed first on the primary and then on the key secondary endpoints 1 and 2, separately for each dose comparison to placebo.

Statistical tests on both the primary and the key secondary endpoints will be at the same significance level alpha (0.025), Key secondary endpoint 1 will be tested only if the test on the primary endpoint is statistically significant. Testing within a dose level will be stopped if the test on the primary endpoint could not reject null hypothesis for the compound dose level at significance level of 0.025.

Key secondary endpoint 2 will be tested only if the test on the key secondary endpoint 1 is statistically significant. Testing within a dose level will be stopped if the test on the key secondary endpoint 1 could not reject null hypothesis for the compound dose level at significance level of 0.025.

Significance level 0.025 is Bonferroni adjusted significance level obtained by dividing the original significance level 0.05 by the number of comparisons within endpoint family between each compound dose level and placebo, i.e. the adjusted significance level is 0.025 (0.05 divided by 2).

The null hypothesis is that there is no difference in survival distribution of the time to beginning of symptom relief defined by the PGI-C as at least “a little better” for 2 time points in a row within 12 hours of the first study drug administration (no difference between each dose of the compound group versus placebo group) versus the alternative hypothesis that the survival distributions are different (each of the compound dose groups versus placebo).

H₀:tₖ−tₚ=0,

where tₖ is the time to “a little better” or higher rating of HAE attack following the compound dose treatment and tₚ is the time to “a little better” or higher rating of HAE attack following placebo.

Hₐ:tₖ−tₚ≠0.

The primary endpoint will be analysed using Gehan Score Transformation test proposed by Feingold and Gillespie (1996) (Feingold M, Gillespie B W. Cross-over trials with censored data. Stat Med. 1996; 15:953-67) for crossover trials with censored data.

The number of patients with the primary endpoint event will be summarized by frequencies and survival estimates, the summaries will be presented by treatment.

HAE attack severity will be assessed on the PGI-S 5-point Likert scale scored as none, mild, moderate, severe, and very severe. A decrease in severity is defined as any change to any less severe level post baseline, than the score reported at baseline.

Key secondary endpoints will be analyzed with the same approach as primary endpoint analysis method (Gehan Score Transformation test). Key secondary endpoints will be tested according to the fixed sequence closed testing procedure. Key secondary endpoints will be summarized by frequencies and survival estimates; the summaries will be presented by treatment.

Subgroup analyses of the primary and key secondary efficacy endpoint will be performed by primary attack location at HAE attack onset, gender, age, Baseline severity, region, and number of doses received. Frequencies and survival estimates will be presented for each subgroup. Subgroups by time of attacks may also be investigated.

Analysis of the primary and key secondary efficacy endpoints will be performed on the FAS and the prophylaxis FAS.

Secondary endpoints will be summarized by frequencies and percentages and presented by treatment.

All time-to-event endpoints will be summarized by frequencies of the events and Kaplan-Meier estimates or survival estimates as appropriate. The summaries will be presented by treatment.

For the exploratory endpoints baseline- and time-adjusted AUCs of the GA-NRS over time from 0 hour (pre-dose) up to 12 hours and up to 24 hours or up to the last time point prior to the time of conventional on-demand treatment use, whichever occurs first will be derived by linear trapezoidal rule. AUCs will be summarized descriptively. A statistical comparison of the AUCs between treatments will be performed using a mixed effects analysis of variance with fixed effects of treatment, sequence, and period and patient nested within sequence as a random effect.

Safety

Safety analyses will be performed by treatment group using the safety set. Safety endpoints include AE, clinical laboratory assessments, vital signs, and ECG findings.

Adverse events and serious adverse events (SAEs) recorded during the trial will be summarized by system organ class, preferred term, and treatment. Adverse events and medical history will be coded using the most current version of MedDRA.

Frequencies and percentages of patients with treatment-emergent adverse events (TEAEs), serious TEAEs, and TEAEs causing premature discontinuation will be provided by treatment group.

Patient listings of all AEs will be provided as well as listings of deaths, SAEs, and AEs leading to discontinuation.

For physical examination, ECG, vital signs, and laboratory variables (measured by the central laboratory), the number and percentage of patients with normal or abnormal results will be presented at each scheduled visit by sequence. Descriptive statistics for continuous variables will be provided at scheduled visits together with a summary of changes from baseline for each parameter by sequence.

### Example 8—Phase 1 Multiple Dose Study in Healthy Adult Subjects

Aim: To evaluate the safety, tolerability, pharmacokinetics, and the change from baseline in QTc following administration of the compound formulated as 100 mg film coated tablets in healthy adult subjects.

Primary Objective:
To investigate the safety and tolerability of multiple doses of the compound.

Secondary Objectives:
To investigate the pharmacokinetics (PK) of multiple doses of the compound.
To evaluate the effects of the compound on ECG parameters, including concentration-QTc relationship, following administration of the compound 100 mg Film Coated Tablets (KalVista Pharmaceuticals) to healthy adult subjects.

Exploratory Objectives:
To investigate the pharmacodynamics (PD) of multiple doses of the compound.

Methods:

This was a phase 1, double-blind, placebo-controlled, multiple-dose, multiple-cohort study to evaluate safety and tolerability of the compound as well as of the ECG effects of the compound formulated as 100 mg Film Coated Tablets in healthy adult male and female subjects.

Four (4) cohorts were evaluated. Cohorts 1, 2 and 3 included 8 subjects each. Cohort 4 included 18 subjects. Every attempt was made to include an equal number of male and female subjects in each cohort.

During the study, oral doses of 600 mg of the compound as Film Coated Tablets (six 100 mg tablets) or 6 matching placebo tablets were administered once every 8 hours (Cohort 1) every 4 hours (Cohort 2), or every 2 hours (Cohort 3 and 4) to healthy adult male and female subjects up to a total dose of 1800 mg. In Cohorts 1, 2 and 3, 6 subjects received the compound as 100 mg Film Coated Tablets and 2 subjects received the placebo for a total of 8 subjects per cohort. In Cohort 4, 12 subjects received the compound as 100 mg Film Coated Tablets and 6 subjects received the placebo for a total of 18 subjects.

Progression from Cohort 1 to Cohort 2 and Cohort 2 to Cohort 3 occurred after review of the safety data (labs, vital signs, safety ECGs, and adverse events) captured during the conduct of Cohort 1 and Cohort 2. Progression to Cohort 4 occurred after review of the safety data and pharmacokinetic data from Cohort 3. The pharmacokinetic data from Cohort 3 was reviewed to ensure that the Cmax of the 3rd dose is high enough to support the evaluation of the change in the QTc interval from baseline.

A Holter monitor was attached to each subject in order to continuously record ECGs. The monitor was attached 1 hour before the first dose and remained attached until after the final blood sample collection. The electrodes for the Holter monitor were checked by a member of the clinic staff at appropriate intervals to ensure they were attached.

Blood samples were collected at pre-dose, at intervals after the first dose, and at intervals over 24 hours after the final (third) dose (40 hours from the initial dose in Cohort 1, 32 hours from the initial dose in Cohort 2, 28 hours from the initial dose in Cohorts 3 and 4) in each cohort. Subjects were confined to the clinical facility from at least 10 hours before dosing until after the final blood sample collection in each study cohort and returned to the clinic 5 to 7 days after the final dose for safety evaluations.

The pharmacokinetics of the compound were measured by a fully validated analytical procedure and the pharmacodynamic effect on plasma kallikrein inhibition enzyme activity was evaluated by an exploratory pharmacodynamic assessment.

Statistical analysis was performed to evaluate the relationship between plasma drug concentrations and the change from baseline in ECG effects of the test formulation.

Treatment Administration

Cohort 1

The subjects received the test or placebo treatment every 8 hours over a 16-hour period (3 administrations of: 6×100 mg of the compound as 100 mg Film Coated Tablets or placebo dose administrations at 0, 8, and 16 hours, total dose of 1800 mg of the compound or placebo) according to a two-treatment randomization schedule under direct observation. Each dose was administered with 240 mL of room temperature water. Subjects were instructed to swallow the tablets whole without chewing or biting. Any subject who bit or chewed the tablets was dropped from the study. Immediately after dosing a mouth check was performed

Cohort 2

The subjects received the test or placebo treatment every 4 hours over an 8-hour period (3 administrations of: 6×100 mg of the compound as 100 mg Film Coated Tablets or placebo dose administrations at 0, 4, and 8 hours, total dose of 1800 mg of the compound or placebo) according to a two-treatment randomization schedule under direct observation. Each dose was administered with 240 mL of room temperature water. Subjects were instructed to swallow the tablets whole without chewing or biting. Any subject who bit or chewed the tablets was dropped from the study. Immediately after dosing a mouth check was performed to ensure that the tablets were swallowed whole without chewing or biting.

Cohort 3 and 4

The subjects received the test or placebo treatment every 2 hours over a 4-hour period (3 administrations of: 6×100 mg of the compound as 100 mg Film Coated Tablets or placebo dose administrations at 0, 2, and 4 hours, total dose of 1800 mg of the compound or placebo) according to a two-treatment randomization schedule under direct observation. Each dose was administered with 240 mL of room temperature water. Subjects were instructed to swallow the tablets whole without chewing or biting. Any subject who bit or chewed the tablets were dropped from the study. Immediately after dosing a mouth check was performed to ensure that the tablets were swallowed whole without chewing or biting.

All subjects fasted (except water) for at least 8 hours before the first dosing. After initial dosing, subjects continued to fast until at least 6 hours after the first dose.

Method of Assigning Subjects to Treatment Groups:

Cohort 1, 2 and 3

Subjects were randomized such that 6 subjects received the test product and 2 subjects received the placebo. As a safety measure, a sentinel dosing scheme was incorporated for each cohort, in which one subject will receive the test product and one subject received the placebo product followed by the remainder of the cohort.

Cohort 4

Subjects were randomized such that 12 subjects received the test product and 6 subjects received the placebo.

The randomization schedule was generated prior to the first dosing cohort using SAS*, Version 9.4 or higher.

Results:

No serious adverse events were reported during the study and no subjects were discontinued because of an AE. All reported adverse events were considered “mild” in severity and had an outcome of “recovered/resolved” at the end of the study.

No clinically relevant effects on the studied ECG parameters were identified.

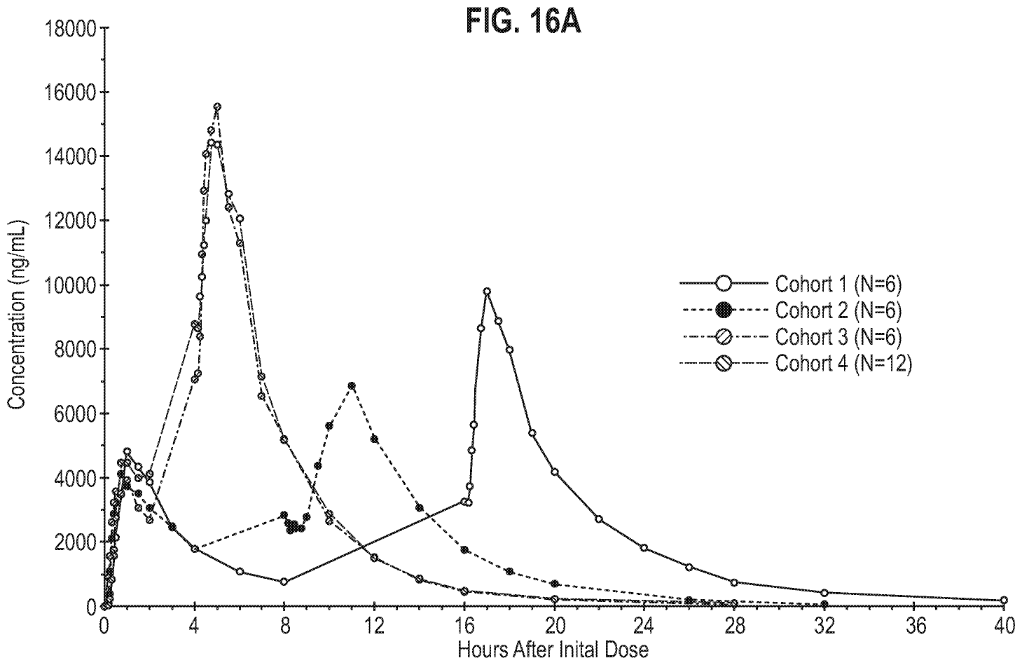

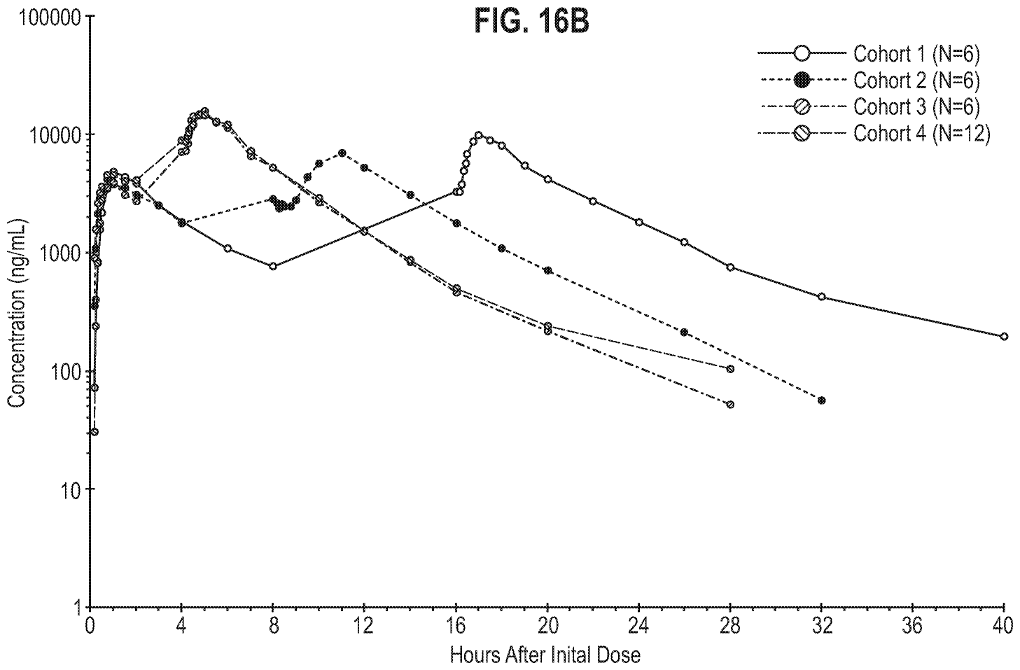

FIG. 16A shows the mean plasma concentrations of the compound of Formula A after the initial dose for each cohort.

FIG. 16B shows the mean plasma concentrations (semi-logarithmic scale) of the compound for formula A for each cohort.

These data demonstrate that the compound of Formula A has a pharmacokinetic profile suitable for oral administration when administered in multiple dosage amounts. The results further suggest that the compound of Formula A can be dosed safely at regular intervals.

1. A method for treating an attack of hereditary angioedema (HAE) on-demand comprising: orally administering the compound of Formula A or a pharmaceutically acceptable salt and/or solvate thereof to a patient in need thereof on-demand, wherein the treatment reduces the need for a second treatment of the HAE attack within 4 hours of taking the compound of Formula A or a pharmaceutically acceptable salt and/or solvate thereof,

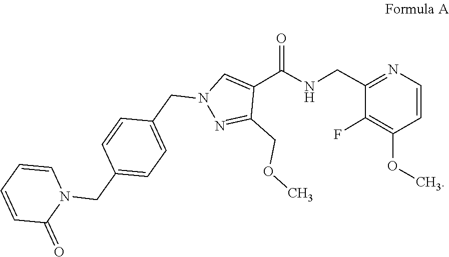 [SMILES: COCc1nn(Cc2ccc(Cn3ccccc3=O)cc2)cc1C(=O)NCc1nccc(OC)c1F]

2. The method according to claim 1, wherein the treatment reduces the need for a second treatment of the HAE attack within 12 hours of taking the compound of Formula A or a pharmaceutically acceptable salt and/or solvate thereof.

3. The method according to claim 1, wherein the treatment reduces the need for a second treatment of the HAE attack within 24 hours of taking the compound of Formula A or a pharmaceutically acceptable salt and/or solvate thereof.

4. The method according to claim 1, wherein the treatment avoids the need for a second treatment of the HAE attack within 4 hours of taking the compound of Formula A or a pharmaceutically acceptable salt and/or solvate thereof.

5. The method according to claim 4, wherein the treatment avoids the need for a second treatment of the HAE attack within 12 hours of taking the compound of Formula A or a pharmaceutically acceptable salt and/or solvate thereof.

6. The method according to claim 5, wherein the treatment avoids the need for a second treatment of the HAE attack within 24 hours of taking the compound of Formula A or a pharmaceutically acceptable salt and/or solvate thereof.

7. The method according to claim 1, wherein the patient is not administered a second treatment of the HAE attack within 4 hours of taking the compound of Formula A or a pharmaceutically acceptable salt and/or solvate thereof.

8. The method according to claim 7, wherein the patient is not administered a second treatment of the HAE attack within 12 hours of taking the compound of Formula A or a pharmaceutically acceptable salt and/or solvate thereof.

9. The method according to claim 8, wherein the patient is not administered a second treatment of the HAE attack within 24 hours of taking the compound of Formula A or a pharmaceutically acceptable salt and/or solvate thereof.

10. The method according to claim 1, wherein the attack is resolved without administering a second treatment to the patient.

11. A method for treating an attack of hereditary angioedema (HAE) on-demand comprising: orally administering the compound of Formula A or a pharmaceutically acceptable salt and/or solvate thereof to a patient in need thereof on-demand, wherein symptom relief of the attack starts within 3 hours of administering the compound of Formula A or a pharmaceutically acceptable salt and/or solvate thereof,

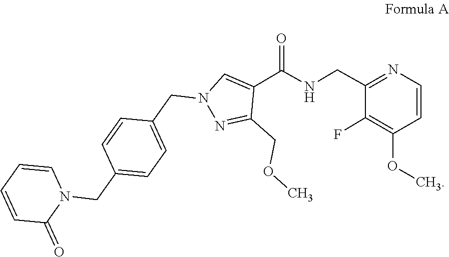 [SMILES: COCc1nn(Cc2ccc(Cn3ccccc3=O)cc2)cc1C(=O)NCc1nccc(OC)c1F]

12. The method according to claim 11, wherein “symptom relief” means that the attack is rated as “a little better” or higher for two consecutive time points when assessed according to the 7-point transition questions (7TQ).

13. The method according to claim 12, wherein the “two consecutive time points” are about 30 minutes apart.

14. The method according to claim 11, wherein the symptom relief of the attack starts between about 1.5 and 3.0 hours after administering the compound of Formula A or a pharmaceutically acceptable salt and/or solvate thereof.

15. A method for treating an attack of hereditary angioedema (HAE) on-demand comprising: orally administering the compound of Formula A or a pharmaceutically acceptable salt and/or solvate thereof to a patient in need thereof on-demand, wherein symptom relief of the attack starts between about 1.5 and 9 hours after administering the compound of Formula A or a pharmaceutically acceptable salt and/or solvate thereof,

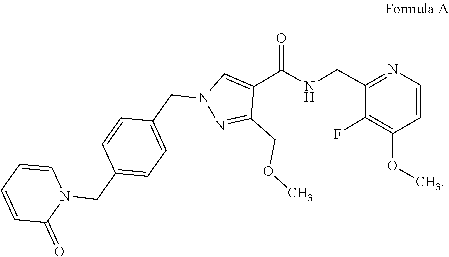 [SMILES: COCc1nn(Cc2ccc(Cn3ccccc3=O)cc2)cc1C(=O)NCc1nccc(OC)c1F]

16. The method according to claim 15, wherein “symptom relief” means that a 50% reduction in composite VAS score occurred for three consecutive time points.

17. The method according to claim 16, wherein the “three consecutive time points” are about 30 minutes apart within 0-4 hours after administration of the compound of Formula A or a pharmaceutically acceptable salt and/or solvate thereof and, if necessary, about 1 hour apart within 4-12 hours after administration of the compound of Formula A or a pharmaceutically acceptable salt and/or solvate thereof.

18. The method according to claim 15, wherein the symptom relief of the attack starts between about 3 and 9 hours after administering the compound.

19. A method for treating an attack of hereditary angioedema (HAE) on-demand comprising: orally administering the compound of Formula A or a pharmaceutically acceptable salt and/or solvate thereof to a patient in need thereof on-demand, wherein the treatment halts the progress of the attack such that the symptoms of the attack do not worsen within a 12 hour period after administration of the compound of Formula A or a pharmaceutically acceptable salt and/or solvate thereof,

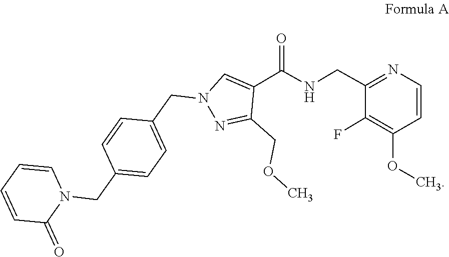 [SMILES: COCc1nn(Cc2ccc(Cn3ccccc3=O)cc2)cc1C(=O)NCc1nccc(OC)c1F]

20. The method according to claim 19, wherein “do not worsen” means that the attack did not worsen by one level or more on the 5LS within the 12 hours of administering the compound.

21. The method according to claim 19, wherein the treatment halts the progress of the attack such that the symptoms of the attack do not worsen within a 24 hour period after administration of the compound of Formula A or a pharmaceutically acceptable salt and/or solvate thereof.

22. The method according to claim 21, wherein “do not worsen” means that the attack did not worsen by one level or more on the 5LS within the 24 hours of administering the compound.

23. A method for treating an attack of hereditary angioedema (HAE) on-demand comprising: orally administering the compound of Formula A or a pharmaceutically acceptable salt and/or solvate thereof to a patient in need thereof on-demand, wherein the treatment improves the severity of the attack to “none” (when assessed according to the 5LS),

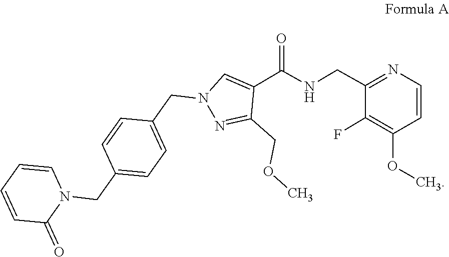 [SMILES: COCc1nn(Cc2ccc(Cn3ccccc3=O)cc2)cc1C(=O)NCc1nccc(OC)c1F]

24. The method according to claim 23, wherein the severity improves to “none” within 24 hours of administering the compound.

25. The method according to claim 23, wherein the severity improves to “none” within 12 hours of administering the compound.

26. The method according to claim 23, wherein the severity improves to “none” within 6 hours of administering the compound.

27. The method according to claim 1, wherein the compound of Formula A or a pharmaceutically acceptable salt and/or solvate thereof is for use in treating an attack of hereditary angioedema (HAE) on-demand and is orally administered on-demand upon recognition of a symptom of an HAE attack.

28. The method according to claim 27, wherein the symptom of an HAE attack recognised is at least one of: swelling of tissues; fatigue; headache; muscle aches; skin tingling; abdominal pain; nausea; vomiting; diarrhoea; difficulty swallowing; hoarseness; shortness of breath; or mood changes.

29. The method according to claim 28, wherein the compound of Formula A or a pharmaceutically acceptable salt and/or solvate thereof is orally administered on-demand within 1 hour of the symptom of an HAE attack being recognised.

30. The method according to claim 29, wherein the compound of Formula A or a pharmaceutically acceptable salt and/or solvate thereof is orally administered on-demand within 30 minutes, within 20 minutes, within 10 minutes, or within 5 minutes of the symptom of an HAE attack being recognised.

31. The method according to claim 30, wherein the compound of Formula A or a pharmaceutically acceptable salt and/or solvate thereof is orally administered on-demand in the prodromal phase of an HAE attack.

32. The method according to claim 31, wherein the symptom recognised is at least one of: a slight swelling, abdominal pain or reddening of the skin.

33. The method according to claim 31, wherein the symptom recognised is Erythema marginatum.

34. The method according to claim 1, wherein the treatment shortens the duration of the HAE attack.

35. The method according to claim 1, wherein the compound or a pharmaceutically acceptable salt and/or solvate thereof is administered as an oral dosage form comprising: (i) the compound or a pharmaceutically acceptable salt and/or solvate thereof, and (ii) a pharmaceutically acceptable excipient.

36. The method according to claim 35, wherein the oral dosage form is a tablet comprising microcrystalline cellulose as a diluent, croscarmellose sodium as a disintegrant, polyvinyl pyrrolidone as a binder, and optionally magnesium stearate as a lubricant.

37. The method according to claim 1, wherein the patient is administered about 600 mg of the compound of formula A.

38. The method according to claim 37, wherein each dosage amount is administered as two tablets each comprising about 300 mg of the compound of formula A.
